(19) United States
(12) Patent Application Publication
(10) Pub. No.: US 2005/0222515 A1
(43) Pub. Date: Oct. 6, 2005

(54) Cardiovascular sound signature: method, process and format

(75) Inventors: Vladimir Polyshchuk, (US); Vladimir Kudriavtsev, (US)

Correspondence Address: Vladimir Polyshchuk, 29 Downing Ct., Exeter, NH, 03833, US

(73) Assignee: Biosignetics Corporation

(21) Appl. No.: 11/064,240

(22) Filed: Feb. 23, 2005

Related U.S. Application Data
(60) Provisional application No. 60/546,742, filed on Feb. 23, 2004.

(51) Int. Cl. A61B 5/02

(52) U.S. Cl. 600/528

(57) ABSTRACT

Devices, systems and methods of analyzing patient's heart, which are particularly focused on noninvasive techniques of interpreting cardiovascular sounds allow cost-effective and quick diagnosis at the early stages of cardiac dysfunctions. They are equally applicable to adult and pediatric patients. The invention recognizes that effective detection of abnormal cardiovascular sounds and heart lesions can be significantly enhanced by techniques, systems and computer media format that allow to present cardiovascular sounds in time and frequency while keeping signal resolution equally strong in both directions. Invention describes the format and method of constructing it to present the patient's cardiovascular sounds for the detection, identification and prognostication of the heart diseases. A truly unique and novel characteristic of the heart energy signature is its self-referencing feature. Additional benefits include the ability to evaluate heart changes as treatment or disease progresses and to provide unified format for the electronic storage of heart auscultation and cardiac exam findings.

A portion of the disclosure of this patent document contains material, which is subject to copyright protection. The copyright owner has no objection to the facsimile reproduction of the patent disclosure as it appears in the Patent and Trademark Office patent file or records, but otherwise reserves all copyright rights whatsoever.

### BACKGROUND OF THE INVENTION

I. Field of the Invention

The present invention generally relates to devices, systems and methods for diagnosing and/or treating of the heart diseases.

In the particular embodiment, the invention provides techniques for converting normal and abnormal cardiovascular sounds into self-referencing visual images; format for storage and display of the signature of the heart sound energy in the electronic media and a method of processing of this signature to derive substantial clinical conclusions. Cardiovascular sounds include heart sounds, Korotkoff sounds, arterial, carotid artery, jugular veins, coronary or venous pulses. Sounds can be collected externally to the body or internally using electro-acoustic, piezoelectric, skin vibration and ultrasound methods.

Presently, the use of a fingerprint, an iris measurement and the DNA to capture very reliable individual identifying characteristics of a patient are well established. It is said that they serve as the unique biometric features of a human. Recently, the biometric features of a patient were expanded to include the patient's digital images obtained by MR scanner and a CT tomograph. The defining characteristic of the biometric features is their static or slowly changing nature. For example, they can slowly change over the life of a patient due to natural growth or disease. The biometric feature does not have to be a static in nature to successfully characterize an individual. A good example of the dynamic or a fast changing biometric feature is a human voice. Although the same individual can vary his or her voice widely, it still can be used to identify that individual. The present invention further expands the class of the individual biometric features to include the signature of the cardiovascular (heart) sound energy. For the purposes of this patent we extend the definition of the heart sound to include all the sounds that can be heard or otherwise detected and are originated from the cardiovascular tract.

Many heart diseases can cause changes in cardiovascular sounds and produce additional murmurs before other signs and symptoms appear. Hence, auscultation (the interpretation by a physician of cardiovascular sounds) and the phonocardiogram (PCG) are two fundamental tools in the diagnosis of heart diseases. The skill to detect and diagnose the heart problem by auscultation and phonocardiogram can take years to acquire and refine. In essence, auscultation relies on:
hearing many cardiovascular sounds, including short-term changes in the them
correctly determining the correspondence of the primary cardiovascular sounds
detecting the correct sequence of the cardiovascular sounds that are closely spaced in time
distinguishing the cardiovascular sounds from the irrelevant and always present background noise

It is documented in the medical literature that primary care physicians, residents and medical students are not adequately trained to perform the auscultation. In many cases two experienced cardiologists can often arrive at different conclusions. In addition, the human ear cannot hear many cardiovascular sounds emitted above and below of the audible frequency range, which, for an average human, is between 50 Hz and 14,000 Hertz (“Hz”). Many cardiovascular sounds are below 50 Hz frequency. Human ear also has the ability to phase certain signals out and interprets higher pitch sounds “louder” in comparison with lower pitch sounds that dominate the heart sound spectrum.

Recently, computer-aided auscultation methods were proposed and implemented by the Inovise Inc., and Zargis Medical Systems. Unfortunately, these methods and systems do in fact eliminate a physician out of the bed-side auscultation process, and rely on statistical or empirical waveform analysis to determine certain patterns in the sounds. These methods do not perform well with noisy signals, do not work with the stethoscope, and can not be utilized by the individual doctor in conjunction with the stethoscope during the act of the cardiac exam. Their diagnosis premises require extensive validation and, for this reason, are not currently acceptable for patient care reimbursement by the Medicare and major HMO. See, for example “Acoustic Heart Sound Recording and Computer Analysis”, (August 2004), Aetna Inc. Clinical Policy Bulletin #0692, (consisting of 2 pages).

The existing methods and techniques for manual and computer-aided auscultation have significant disadvantages. New auscultation interpretation methods need to be developed that can work in conjunction with the physician's stethoscope or in conjunction with any other sound collecting sensor or device. The main objective of such a method and system should be to empower the physician to perform cardiac exam more effectively, reduce time and cost of the diagnosis and to considerably improve patient access to the healthcare system.

II. Related Art

The following U.S. Patents provide useful background for the current Invention and are herein incorporated by reference: U.S. Pat. Nos. 5,218,969; 5,213,108; 5,025,809, 5,860,933 and 5,971,936.

To overcome the challenges of the auscultation, the technique of phonocardiography has been used to visualize the cardiovascular sounds. As indicated by Bredesen in U.S. Pat. No. 5,213,108 and Johnson in U.S. Pat. No. 5,025,809, the phonocardiograph, the device used in phonocardiography, usually displays a time plot of the heart sounds called PCG signal. Phonocardiograph can also be enhanced with the spectral processing of the cardiovascular sounds as indicated in work done by Bennett in U.S. Pat. No. 4,967,760, Mohler in U.S. Pat. No. 6,053,872 and Kudriavtsev, V. V., et al. (“Hemodynamic Pressure Instabilities and their Relation to Heart Auscultation”, Proceedings of ASME PVP Division Conference, 5th International Symposium on Computational Technologies for Fluid/Thermal/Chemical/Stressed Systems with Industrial Applications, Jul. 25-29, 2004, #PVP2004-3126, ASME PVP Vol. 491-2, pp. 113-122, San Diego/La Jolla, USA).

Mohler in U.S. Pat. No. 4,967,760 defines sonospectrography as the separation and arrangement of the frequency components of acoustic signals in terms of energy or time. Spectrograms were originally developed by McCusik, V. A. et al. (“On Cardiovascular Sound”, 1955, Circulation 11:849) and later used by Winer, D. E. et al. (“Heart sound analysis: A three dimensional approach”, Am. J. Cardiol. 16:547, (1965)). Brief review on the subject is given by Tavel M. E. (“Clinical Phonocardiography and External Pulse Recording”, 2nd Ed, Year Book Medical Publishers, p. 19, (1972)). Acoustic spectrograms have not been actively used in the practice of medical care due to their inherent disadvantages. They are based on short term window Fast Fourier Transform (“FFT”) or it's another derivative called Gabor transform. They are not mathematically unique, they are window dependent techniques and one can easily generate very different looking image representations for the very same heart sound. They also suffer from the inability to provide simultaneous resolution in both time and frequency. As a result, the final time frequency dependencies can be inaccurate, difficult to interpret and are prone to errors. Most recent use of the spectrograms was shown in the textbook by T. A. Don Michael (“Auscultation of the Heart, McGraw Hill, pp. 1-404, (1998)) and mainly for the illustration purposes. Acoustic spectrograms presented in the book do not provide sufficient frequency resolution and thus add very little towards the separation and understanding of the cardiac events.

Eisenberg et al. in U.S. Pat. No. 4,792,145; Andries in U.S. Pat. No. 4,991,581 and Lund et al. in U.S. Pat. No. 4,679,570 disclose phonocardiography with signal processing and visual output. Semmlow et al. in U.S. Pat. No. 5,036,857 discloses a method of phonocardiography with piezoelectric transducer and recommends against FFT analysis of the heart sounds.

The major problem with all types of the phonocardiograph devices disclosed up to date is that heart sound signals are often contaminated with noise and it is often very hard to differentiate them using visual or computer methods. Displays are often recorded continuously in time and the segmentation and identification of the important components of the cardiovascular sounds are not performed. Thus, the physician faces the challenge to visually determine the components of the cardiovascular sounds. This is not an easy task, and is usually time consuming and error prone.

Detailed visual analysis of the pre-recorded sound is also difficult and subject to error, requires accumulation of the ‘interpretation experiences’, especially difficult in noisy environments (non-clean recordings) or with the lower grade hardware. These noisy environments seem to dominate in many practical settings, including body noise (bowel, breathing, indigestion), emergency room (“ER”) noise and electrical and radio interference noises. Several statistical and deterministic (“matrix”) methods were also developed to differentiate such signals. First such approaches were exemplified by Durnin R. E., et al. (“Heart-sound screening in children”, JAMA, Vol. 203, pp. 111-116, (1968)) and Ninova P. P. et al. (“Automated phonocardiograph screening for heart disease in children”, Cardiology, Vol. 63, pp. 5-13, 1978). In 1993 Bredesen in U.S. Pat. No. 5,218,969 suggested similar “matrix” semi-empirical methods that are in part based on the heart physiology. Most latest works included neural networks and are presented by Watrous et al. U.S. Pat. No. 6,572,560 and U.S. Pat. No. 6,629,937 and DeGroff et al. U.S. Pat. No. 6,629,937 and DeGroff, C. G. et al. (“Artificial Neural Network-BasedMethod of Screening Heart Murmurs in Children”, Circulation, 2001;103:2711-2716). All of them require extensive calibration or rely on empirical statistics and do not provide any fundamental explanation of the diagnostic solution or easy interpretation of the patient's data.

As exemplified by Rangayyan R. M. and Lehner R. J., “Phonocardiogram signal analysis: a review”, Crit. Rev. Biomed. Eng., Vol. 15, pp. 211-236, (1987)) cardiovascular sounds are intensely been studied by the researchers in the field of the signal processing. In recent decade, the interest in the time-frequency signal representation spurred the research in its application to various fields, including the heart sound analysis.

Xu J. et al. (“Nonlinear transient chirp signal modeling of the aortic and pulmonary components of the second heart sound”, IEEE Trans. Biomed. Eng., Vol. 47, No. 7, pp. 1328-1334, (2000) and Wood J. C. et al. (“Time-Frequency Transforms: A New Approach to First Heart Sound Frequency Dynamics,” IEEE Trans. Biomed. Eng., vol. 39, No. 7, pp. 730-740, (1992)) studied the recorded and computer simulated S1 and S2 components cardiovascular sounds from animals (dogs and pigs) using Wigner-Ville Distribution and the Binomial Transform.

Chen D., et al. (“Time-frequency analysis of the first heart sound. Part 1: Simulation and analysis”, Medical & Biological Engineering & Computing, Vol. 35, pp. 306-310, (1997)) have utilized simulated S1 heart sound to compare with S1 recorded from dogs and humans using short-time Fourier Transform (STFT).

Tranulis C., et al. (“Estimation of pulmonary arterial pressure by a neural network analysis using features based on time-frequency representations of the second heart sound”, Medical & Biological Engineering & Computing, Vol. 40, pp. 205-212, (2002)) have studied S2 heart signal obtained from laboratory animals (pigs) utilizing Smoothed Pseudo Wigner-Ville Distribution (SPWVD) that is independently available from commercial software Matlab/Simulink distributed by MathWorks Inc.

Bentley P. M., et al. (“Time-frequency and time-scale techniques for the classification of native and bioprosthetic heart valve sounds”, IEEE Trans. Biomed. Eng., Vol. 45, No. 1, (1998)) gives an example of the heart sound recorded form the patient with bioprosthetic valves and corresponding various time-frequency transforms (short-time Fourier Transform (STFT), Wigner distribution, Choi-Williams distribution, continuous wavelet transform (CWT) and discrete wavelet transform (DWT)).

The advancements in the field of the digital signal processing of the phonocardiograms are periodically reviewed.

Rangayyan R. M. and Lehner R. J. (“Phonocardiogram signal analysis: a review”, Crit. Rev. Biomed. Eng., Vol. 15, pp. 211-236, (1987)) point out that the heart sound signal has much more information than can be assessed by the human ear or by visual inspection of the signal tracings on paper as currently practiced.

It was found by Obaidat M. S. (“Phonocardiogram signal analysis: techniques and performance comparison”, Journal of Medical Engineering Technology, Vol. 17, pp. 221-227, (1993)) that the spectrogram obtained with short-time Fourier Transform (STFT) cannot detect the first four sounds of the PCG signal. According to the above cited article the Wigner distribution can provide time-frequency characteristics of the PCG signal, but with insufficient diagnostic information S2 components of the heart sound A2 and P2 are not detectable with the spectrogram or Wigner distribution.

Durand L. G. and Pibarot P. (“Digital signal processing of the phonocardiogram: review of the most recent advancements”, Crit. Rev. Biomed. Eng., Vol. 23, pp. 163-219, (1995)) noted that new time-frequency transforms have the potential to better understand the genesis and transmission of cardiovascular sounds and murmurs.

All of the above referenced analyses have utilized Cohen class time-frequency methods (Wigner, pseudo Wigner Ville, Binomial, and Choi-Williams distributions) to study cardiovascular sounds. However, they have only utilized a small segment of a single heart beat (for example S1 or S2 sound components), and were not including prolonged time intervals in their studies. Prolonged time intervals that are at least one heart beat long or contain several heart beats are required to properly reflect on heart sound changes during the inspiration-expiration cycle. Thus, the above referenced articles are of limited utility for the practical diagnosis. Stankovic L. and Djurovic I. (“A Note on” An Overview of Aliasing Errors in Discrete-Time Formulations of Time-Frequency Representations”, IEEE Trans. on Signal Proc., Vol. 49, No. 1, pp. 257-259, (2001)) were motivated to develop yet another time-frequency transform due to the perception of a poor quality of the Wigner-Ville image due to aliasing and cross-interference terms errors. Wood J. C., et al (“Time-frequency transforms: a new approach to first heart sound frequency dynamics,” IEEE Trans. Biomed. Eng., vol. 39, No. 7, pp. 730-740, (1992)) have concluded that Wigner-Ville method generates considerable errors due to the cross-interference terms and that have rendered a wrongful conclusion that other methods (such as binomial for example) are preferable for the heart sounds analysis. Same wrongful conclusion in favor of the wavelet method over Wigner-Ville method was made by Hayek S. et al. (“Wavelet Processing of Systolic Murmurs to Assist With Clinical Diagnosis of Heart Disease”, Biomedical Instrumentation and Technology, p. 263-270, July/Aug, (2003)).

Wavelet methods, as described by Mertins A. (“Signal Analysis. Wavelets, Filter Banks, Time-Frequency Transforms and Applications”, John Wiley & Sons, (1999)), do provide a robust and quick solution processing capability, however they do not provide visual outputs that can be simply interpreted or understood in terms of time and frequency (pitch). Examples of such outputs are given by Hayek S. et al. (“Wavelet Processing of Systolic Murmurs to Assist With Clinical Diagnosis of Heart Disease”, Biomedical Instrumentation and Technology, p. 263-270, July/Aug (2003)) and they do not provide the way how to interpret wavelet images of the heart sounds.

Related to current invention work was described by Kudriavtsev V. V. and Polyshchuk V. V in the conference article (“Hemodynamic Pressure Instabilities and their Relation to Heart Auscultation”, Proceedings of ASME PVP Division Conference, 5th International Symposium on Computational Technologies for Fluid/Thermal/Chemical/Stressed Systems with Industrial Applications, Jul. 25-29, 2004, #PVP2004-3126, ASME PVP Vol. 491-2, pp. 113-122, San Diego/La Jolla, USA) and in the Biosignetics Corporation product literature and press releases:
“Heart Energy Signature HES Visualization System Version 2”, http://www.bsignetics.com/prod01.htm;
“Phonocardiograph Monitor Main Display”, http://www.bsignetics.com/prod02.htm;
“Digital Stethoscope”, Movers and Shakers, December 2004, New Hampshire Biotechnology Council, www.nhbiotech.com;
“New Heart Research Poised to Help Millions”, July 15^{th} 2004;
“New Millennium Signal Processing Algorithms for Stethoscope Auscultation”, Aug. 3^{rd}, 2004
“State of the Art Heart Auscultation Diagnosis Tools are Highlighted for the Worlds Heart Day”, Sep. 26, 2004;
“Computerized Medical Records—Heart Sounds and Murmurs can be remembered forever”, Jan. 28, 2005.
   Each of these articles is incorporated herein by reference.

In light of the above, it would be desirable to provide improved devices, systems and methods for computerized representation of cardiovascular sounds to aid physicians in the processes of auscultation and diagnosis. The present invention provides such improvements, mitigating and/or overcoming at least some of the disadvantages of known approaches for sound based diagnosing of the heart and cardiovascular systems.

### OBJECTS AND ADVANTAGES

It appears that the difficulty in interpreting time-frequency representations of the cardiovascular sounds facing the researchers and inventors in the field is due to the lack of the defined format (standard) or characteristic signature for the heart sound. The researchers and inventors are lacking a proper basis for comparison of the results of their studies. The situation is reminiscent of the invention of the periodic table of chemical elements by Mendeleev. At the time of his invention, many chemical elements were well-known and studied. However, by proper grouping of the chemical elements into a new representation format, a significant breakthrough in the chemistry and physics was achieved. New patterns of the relationship between the chemical elements became evident, which led to discovery of many new elements and new properties.

Although the above patents and the research publications disclose some forms of the time-frequency representations of the cardiovascular sounds, none of these patents and the publications disclose a method of obtaining a unique signature of the heart and cardiovascular sound and its energy. Also none of these patents and the publications discloses a format to represent, display and to store the signature of the heart sound and the method of obtaining such format. Further, none of the above patents and publications discloses the method to utilize the heart sound energy signatures for the detection, identification, time evolution and prognosis of the heart conditions and their changes.

### SUMMARY OF THE INVENTION

The present invention provides improved devices, systems and methods for performing auscultation of the heart with the help of signal processing techniques based on the theory of system science. The techniques of the present invention are particularly useful for the detection of the characteristic signatures of the heart sound energy typical to congestive heart failure, left ventricular dysfunction, innocent systolic murmurs in children, atrial septal defect, pulmonary stenosis, ventricular septal defect, heart murmur classification, normal and abnormal splits in the cardiovascular sounds, diastolic gallop, atrial sound, quadruple gallop and systolic and diastolic clicks.

The present invention describes the format and method of constructing it to present the patient's cardiovascular sounds for the detection, identification and prognostication of the heart diseases. The main element of the format is the image of the heart sound energy given simultaneously in time and frequency and obtained using sound signal transformation into a joint time-frequency domain. Above image of the heart sound energy is obtained for the time duration period that exceeds or equal to one heart beat of a given living subject. A heartbeat is a single complete pulsation of the heart, consisting of a complete cardiac contraction-relaxation sequence, and produces different sound patterns at different auscultation or sensor locations. By presenting cardiovascular sound in this form, a new biometric signature of the patient is obtained. It is named as the signature of the heart sound energy. A truly novel characteristic of the heart sound energy signature is its self-referencing feature that allows easy qualitative and visual differentiation of the normal heart signature from all the deviant or abnormal heart signatures.

Signature of the heart sound energy can generate several derivative functions that characterize heart energy density spectrum and time varying power. When combined with the original signal of the cardiovascular sound the above indicated elements form the heart energy signature format (HESF).

Heart energy signature format offers a tremendous promise to become de-facto standard of exchanging heart medical data, heart auscultation data and of analyzing these data in cardiac devices, characterization, diagnosis and medical practices, even for home care.

This promise is due to many of heart sound energy signature features:
a) it is self-referencing;
b) it allows precise mathematical and mechanical characterization of heart pulsations in real-time and in stand alone post-test modes;
c) it is mathematically unique;
d) it is easy for the physician, operator or patient to understand and to interpret;
e) it is portable, can be stored and transmitted on/between the variety of media (desktop computers, PDA);
f) organizers, visual cell phones, laptops, palmtops and can be simply printed on the paper)
g) it allows continuous time monitoring to evaluate longer term changes
h) it can be implemented as the algorithm, digital format and software and be always reduced to a graphical image.

The invention provides, in one aspect, the method for coding and estimating the signature of the heart sound energy out of a typical cardiovascular sound dataset. Because of the limitations imposed by the present memory and speed of the computing hardware it is presently not possible to consider limitless length of the corresponding sound dataset. To address above limitations an enhanced method was invented. The preferred embodiment of the method allows implementing size down-sampling and proportional extension of the energy signature time duration using the method of wavelet downsampling or any other method that will not lead to a loss or alteration of the initial signal. An alternative embodiment implements block-by-block computation of the joint time frequency transformation algorithm that allows covering larger time durations using the plurality of short time duration chunks of data.

In the exemplary embodiment of the present method a pseudo Wigner-Ville joint time frequency transformation is utilized to obtain a signature of the heart sound energy for a given time duration. This time duration covers at least one heart beat. Alternative embodiments include all other Cohen class and higher order joint time frequency transformations such as Margenau-Hill, Choi-Williams, or modified pseudo Wigner-Ville as described by Stankovic L. and Djurovic I. (“A Note on “An Overview of Aliasing Errors in Discrete-Time Formulations of Time-Frequency Representations”, IEEE Trans. on Signal Proc., Vol. 49, No. 1, pp. 257-259, (2001)), Stankovic L., (“S class of distributions”, IEEE Proceedings: Vision, Image and Signal Processing, Vol. 144, No. 2, pp. 57-64, (1997)); and Stankovic L., (“L-class of time frequency distributions”, IEEE Signal Processing Letters, Vol. 3, No. 1, pp. 22-25, (1996)).

Since filing the provisional patent U.S. Pat. No. 60/546,742 all the preferred and alternative embodiments were reduced to practice by the inventors in the BSignal software that is currently marketed by the Biosignetics Corporation.

One preferred aspect of the invention includes the method and format for evaluating heart energy signature changes in time on the test by test basis. This is a very important feature that is currently not available in the existing medical practice. Such a feature will lead to a new data and new discoveries that were not possible by any other methods or even by other technology modes.

In another aspect, the invention provides methods and formats for storing and displaying characteristic heart energy signature in the electronic media. Proper storage and display of the relevant information and energy signature components leads to the effective and rapid diagnosis and recognition sequence. We have developed above methods using the experimentation with various cardiovascular sounds representing different diseases, recording equipment and patient types.

In yet another aspect, the present invention provides the system for processing and identification of the new characteristic features of the heart energy signature. This system is based on relating all new characteristic features obtained through the use of signature of the heart sound energy with the existing principles of cardiac auscultation. It is expanded through the use of look-up method of comparing currently obtained heart sound energy signature format against the organized database of similarly structured data using simply visual identification method or using computerized pattern recognition algorithms. It can be implemented in the computer software, training system, database or as a set of manually guided steps.

### BRIEF DESCRIPTION OF THE DRAWINGS

A more complete understanding of the invention may be obtained by reference to the drawings, in which:

FIG. 1 schematically illustrates a generic shape of the visual representation of the heart sound energy signature of a single heart beat

FIG. 2A graphically illustrates typical heart sound representation for several inspiration-expiration cycles

FIG. 2B graphically illustrates enhanced detail of the heart sound consisting of two heart beats

FIG. 2C graphically illustrates heart sound energy signature of the heart sound consisting of two heart beats shown in FIG. 2B

FIG. 3 schematically illustrates heart sound energy signature format

FIG. 4A graphically illustrates the components of the heart sound energy signature format

FIG. 4B graphically illustrates the components of the heart sound energy signature format

FIG. 5A schematically illustrates steps required to obtain a heart sound energy signature

FIG. 5B schematically illustrates steps required to obtain heart sound energy signature using signal down-sampling

FIG. 5C schematically illustrates steps required to obtain heart energy signature using block-by-block processing algorithm

FIG. 6 graphically illustrates the results of the implementation of the heart sound data reduction

FIG. 7A illustrates heart energy signature display option

FIG. 7B illustrates heart energy signature display option

FIG. 7A illustrates heart sound power and signal display option

FIG. 8 schematically illustrates preferred display mode for comprehensive multi-position diagnosis using heart energy signature method

FIG. 9 illustrates various joint time-frequency distributions that can be used to estimate heart sound energy

FIG. 10 schematically illustrates steps required to obtain heart sound energy estimation using pseudo Wigner-Ville transformation

FIG. 11. schematically illustrates heart energy flow format for test to test evaluation

FIG. 12 graphically illustrates principles of visual heart energy signature flow interpretation in three dimensions

FIG. 13A illustrates stand alone application of the heart energy signature device, method and system

FIG. 13B illustrates real time application of the heart energy signature device, method and system

FIG. 14 illustrates heart energy signature display and its use

FIG. 15 illustrates steps required to process heart energy signature for segmentation

FIG. 16 illustrates look up table database approach for the diagnosis

### DETAILED DESCRIPTION OF THE SPECIFIC EMBODIMENTS

While the following description is largely directed to the utilization and processing of the heart sounds, the methods, devices and systems of the present invention may be used for the analysis of a variety of cardiovascular sounds (such as Korotkoff sounds, arterial and venous pulses) and other biological body sounds including indigestion and bowel sounds, baby sounds, fetal and fetal cardiovascular sounds. The invention is well suited to characterize animal cardiovascular and heart sounds, including heart sounds of horses, dogs and cats.

The sounds heard over the cardiac region are produced by the functioning heart. There are four distinct sounds: the first (S1) occurs at the beginning of systole and is heard as a “lub” sound; the second (S2) is produced by the closing of the aortic and pulmonary valves and is heard as a “dub” sound; the third (S3) is produced by vibrations of the ventricular walls when suddenly distended by the rush of blood from the atria; and the fourth (S4) is produced by arterial contraction and ventricular filling. Other cardiovascular sounds may include simpler pulse components.

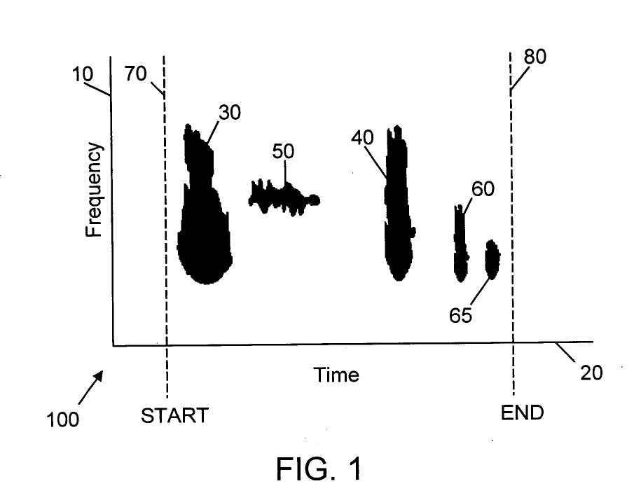

Referring now to FIG. 1, we shall describe the morphology and visual representation of the signature of the heart sound energy for a single heart beat. A generic signature is represented by the graphical image 100 and is shown in time axis 20 and frequency axis 10. It is bounded by left 70 and right 80 temporal boundaries, thus defining its time duration. First heart sound, S1 30, and second heart sound S2 40 are shown schematically, with systolic murmur shown as element 50, third heart sound S3 is shown as element 60 and fourth heart sound S4 is shown as element 65. Depending on a specific heart sound, any additional elements may or may not be present on the signature of the heart sound energy.

Morphologically energy plot 100 consists of the same key elements as the heart sound, i.e. it includes S1 and S2 heart sound normal components superimposed with additional abnormal sound components. S1 30 and S2 40 components of the energy plot are very easy to identify and visualize. Normal heart follows typical “lub-dub” pattern with S1 being “lub” and S2 being “dub”, and thus operator skilled in arts will mostly see S1 30 and S2 40 energy blobs when examining sounds obtained from the normal heart. The additional abnormal sounds are typically characteristic of the diseased heart and may include S3 60 and S4 65 components, systolic and diastolic murmur's components, snaps and clicks components of the signature of the heart sound energy. Each energy blob or component of the energy plot 100 becomes in fact a characteristic element of the heart sound energy signature when shown in a visual format as referred in FIG. 1. Each such component is represented as a dark blob on the image of the heart energy. Heart murmurs are typically seen as several smaller blobs connected together into a “weave” or small blobs located behind the S1 blob 30 or behind the S2 blob 40.

Energy plot has clearly defined temporal borders 70 and 80 that define the beginning and the end of the heart sound energy signature (“HSES”). It shows zones where energy is dissipated on a joint time 20 and frequency 10 plot 100. It is thus clearly showing timing and the instantaneous frequency spectrum of each above referenced heart sound components (30, 40, 50, 60, 65) and allows close monitoring of all significant changes in the heart operation. Such changes may occur due to medical treatment, worsening of conditions, artificial implants, medications, hyper-conditions, nervousness associated with tests and even mental situations/crisis.

Referring to FIG. 1, the heart sound energy signature morphology is self-referencing. That is, it extends itself to an immediate examination without having another reference and allows a visual detection of all key abnormalities associated with the sounds or vibrations emitted by the heart. When energy plot is extended to include several heart beats, the system operator can immediately identify pulse duration (by estimating the duration of repetitive patterns (such as HSES 100), pulse irregularity (by examining HSES time durations).

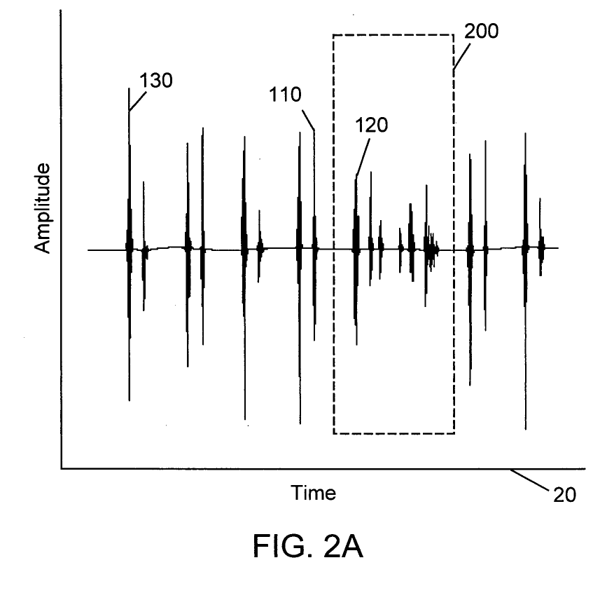

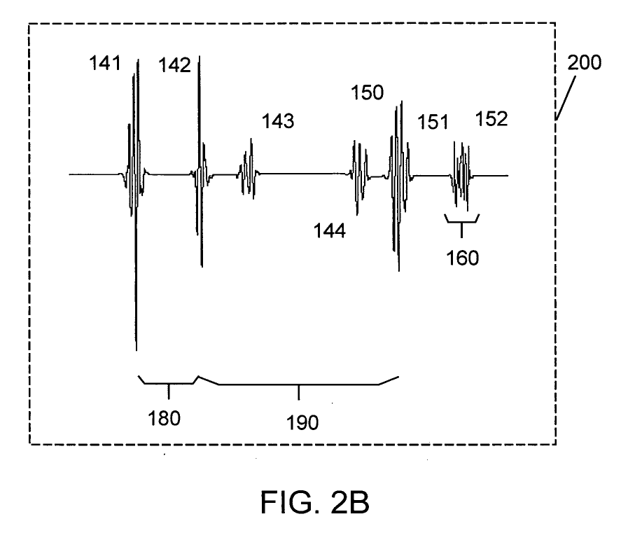

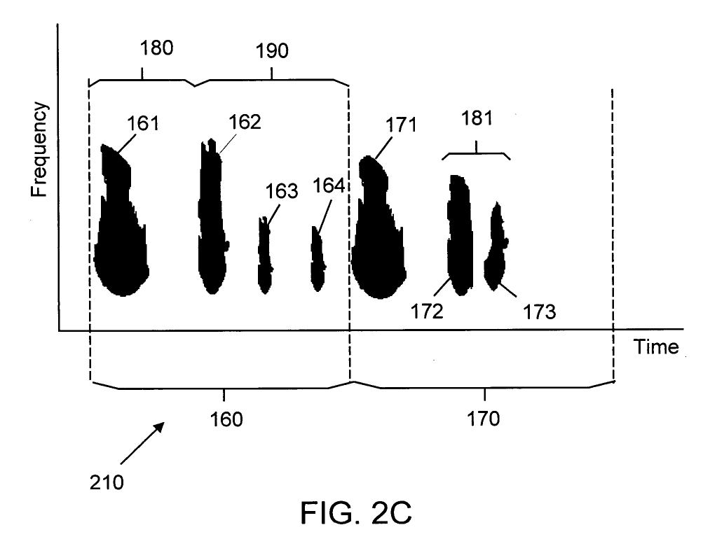

Plot of heart sound amplitude versus time is called phonocardiogram (“PCG”). Referring now to FIG. 2A, the variation of the heart sound amplitude with time is clearly evident. An example of PCG shown in FIG. 2A depicts eight heart beats each consisting of S1 and S2 components. Signal amplitude variation in time is a characteristic heart sound feature that is due to respiration. Signal components 130 and 110 show maximum signal amplitude and according to Tavel, M. E. (“Clinical Phonocardiography and External Pulse Recording, Year Book Medical Publishers, (1972)) are identifiable with expiration. Signal component 120 has lower amplitude and is identifiable with inspiration. Graphical window 200 allows to further zoom into the sound details which are shown on FIG. 2B. They include two neighboring heart beats. This exemplary embodiment includes S1 sound waveform 141, S2 sound waveform 142, S3 sound waveform 143, and S4 sound waveform 144. Systolic time interval 180 is located between the S1 heart sound 141 and S2 heart sound 142, and diastolic time interval 190 is bounded by the S2 heart sound 142 on the left and the next S1 heart sound 150 on the right. S2 heart sound 160 is shown in so-called split format. Elements 151 shows aortic and 152 shows pulmonary component of the S2 heart sound 160. S2 heart sound 142 is not split. Normal heart sound S2 splits with respiration. Splitting of S2 typically happens on inspiration and reaches its maximum time duration when the sound amplitude is minimal.

Referring to FIG. 2C, above described phonocardiographic representation of the cardiovascular sounds 200 is graphically presented as the heart sound energy signature image 210 in time and frequency dimensions. S1 heart sound 161, is followed by the S2 heart sound 162, followed by S3 heart sound 163, followed by S4 heart sound 164. They comprise the first heart beat 160. Systolic period is shown as 180, diastolic period is shown as 190. Second heart beat 181 includes S1 sound 171, and aortic component of S2 sound 172 which is separated by a short time period from the pulmonary component 173, thus, showing S2 sound split on the heart sound energy signature image 210.

The heart sound energy plot 100, as referred on FIG. 1, and characteristic plot 210, as referred on FIG. 2C, represent a unique state of dynamically changing multi-component signal of the heart beat. The plurality of the elements 30, 40, 50, 60, shown on FIG. 1, or the plurality of the elements 161, 162, 163, 164, 171, 172, 173, shown on FIG. 2C, constitutes a visual representation of the signature of the heart sound energy.

Plots referenced on FIG. 1 and on FIG. 2C can utilize various color mapping schemes to indicate changes in the absolute value of the heart sound energy. For the purposes of this patent application we demonstrate black-and-white color scheme. The person skilled in art of image processing can implement many traditional color mapping schemes (gray scale, temperature, red-green-blue, etc.) or non-traditional color mapping schemes to represent heart sound energy signature plot visually.

Hereinafter, the term “heart sound energy signature” will refer to the image or mathematical representation of the image of the time-frequency representation of the heart sound signal that is bounded in time and is at least one heartbeat long in duration. The term “heart sound energy” will refer to the unbounded time intervals or to the time durations that are shorter than a single heart beat.

The above defined heart sound energy signature 100 is intended for use as an individual biometric characteristic for the purposes of heart diagnosis. A new plurality of elements 300 characterizing heart sound can now be assembled. It is comprised from the initial sound signal input 320, heart sound energy signature plot 310 and of various derivative outputs generated from the 310. This new plurality is best fit for the sound based heart diagnosis and will be defined as the heart sound energy signature format 300.

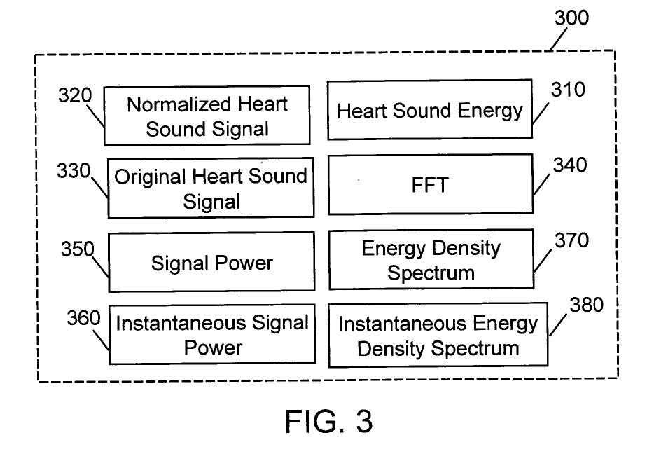

As referred on FIG. 3, the above introduced format 300 utilizes heart sound energy signature time-frequency distribution plot 310, normalized heart sound signal 320, original heart sound as recorded 330, spectral characteristic of the sound 330 obtained via FFT 340, heart sound power 350 and heart energy density spectrum 370 and instantaneous heart sound signal power 360 for any selected frequency and instantaneous density spectrum 380 for any selected moment in time. All above components of the heart sound energy signature format are given for an operator defined time period of the energy signature. This period is at least one heart beat long. The above formulated format 300 must represent a pseudo-periodic portion of the heart sound containing any of the sound components referred on FIG. 1 and FIG. 2C. Let us assume that the heart beat is recorded during the time interval [τ,τ+T] with the measurement instrument being capable to capture the frequency range [f₁,f₂]. As referred on the FIG. 3 an arbitrary heart sound signal can be represented by a number of components identified as 310, 320, 330, 350, 360, 370, 380. The heart energy signature format 300 mathematically includes all of them:
a two-dimensional image matrix representing the distribution of the heart sound energy simultaneously in time and frequency as defined on 310 and displayed as 410
   E=E(t,f), tε[τ,τ+T], fε[f₁,f₂] (1)
a time plot of the normalized heart sound corresponding to the heart sound energy as defined on 320 and displayed as 420
   x=x(t), tε[τ,τ+T], x(t)ε[−1,+1] (2)
a time representation of the original heart sound corresponding to the heart sound energy as defined on 330 and displayed as 430
   x=x(t), tε[τ,τ+T], x(t)ε[A,B], (3)
   where A and B are the lower and upper bounds of the heart sound signal x(t), respectively; a plot of the instantaneous energy of the heart sound, or heart sound power, as defined on 350, 360 and displayed as 450, 460
   P=P(t)tε[τ,τ+T] (4)
a plot of the energy density spectrum of the heart sound, as defined on 370 and 380 and displayed as 470 and 480
   D=|X(f)|², fε[f₁,f₂] (5)
a plot of the energy density spectrum given by a Fourier Transform and computed by FFT on 340 and displayed on 440

$$X ( f ) = \frac{1}{T} {\int }_{τ}^{τ + T} x ( t ) * exp ( - j 2 π ft ) ⅆ t \qquad ( 6 )$$

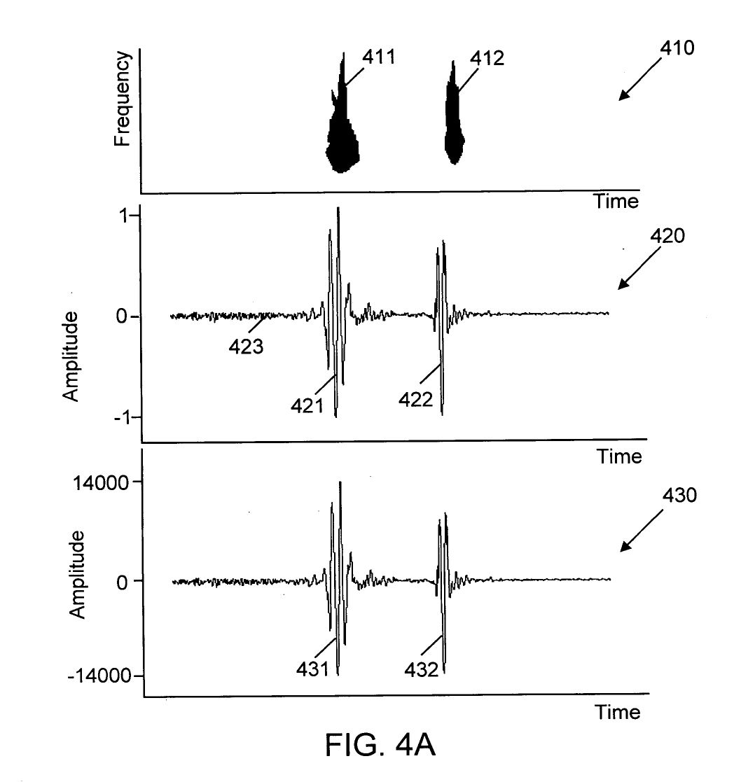

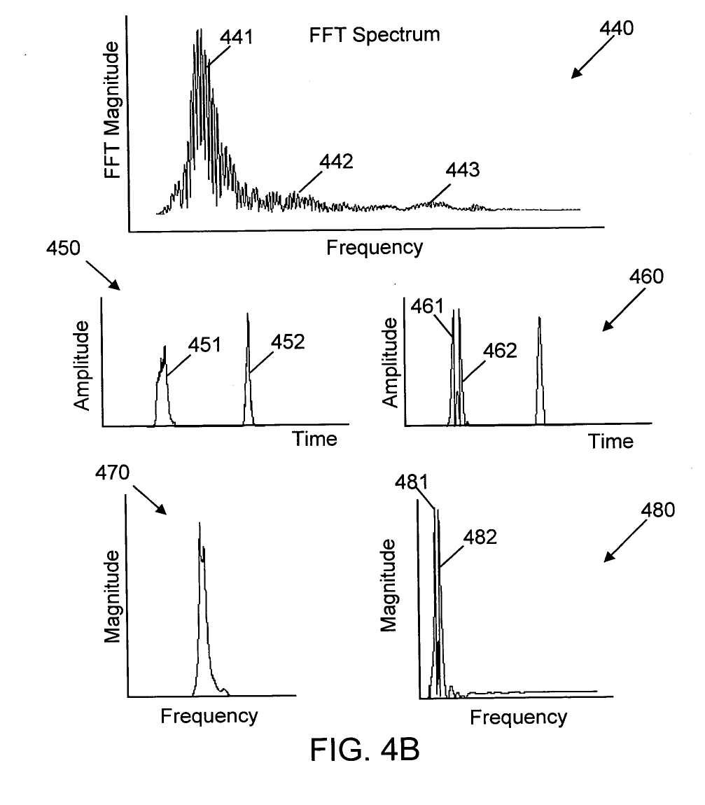

As shown in FIG. 4, plot 410 displays the single heart beat sound with S1 shown as 411 and S2 shown as 412; plot 420 displays normalized phonocardiographic representation of the heart sound with 421 showing S1 and 422 showing S2 and 423 showing low amplitude diastolic noise. Plot 430 displays the original heart sound signal bounded within the signature time limits, with S1 shown as 431 and S3 shown as 432. Plot 440 illustrates FFT spectrum, with 441 indicating frequency of the highest intensity that are usually representative of S1 and S2 sounds and secondary peaks 442 and 443 possibly indicating frequency content of the murmurs. Plot 450 indicates signal power distribution showing S1 as 451 and S2 as 452. Same sound components are shown in the instantaneous power plot 460 that is given for a specific operator selected frequency of interest. It allows to effectively differentiating the presence of various heart sound components on different frequency bands. Here elements 461 and 462 precisely show S1 sound split that is otherwise not visible on the element 451. Plot 470 refers to the integral density spectrum of the signal contained within the bounds of the heart sound energy signature plot 410.

Hereinafter, the term “heart energy signature format” will be referred to as a combination of the “heart energy signature” defined by the Equation (1), original signal defined by the Equation (3), normalized signal defined by the Equation (2), FFT spectrum defined by the Equation (6), instantaneous and integral signal power defined by the Equation (4), instantaneous and integral signal energy density spectrum defined by the Equation (5).

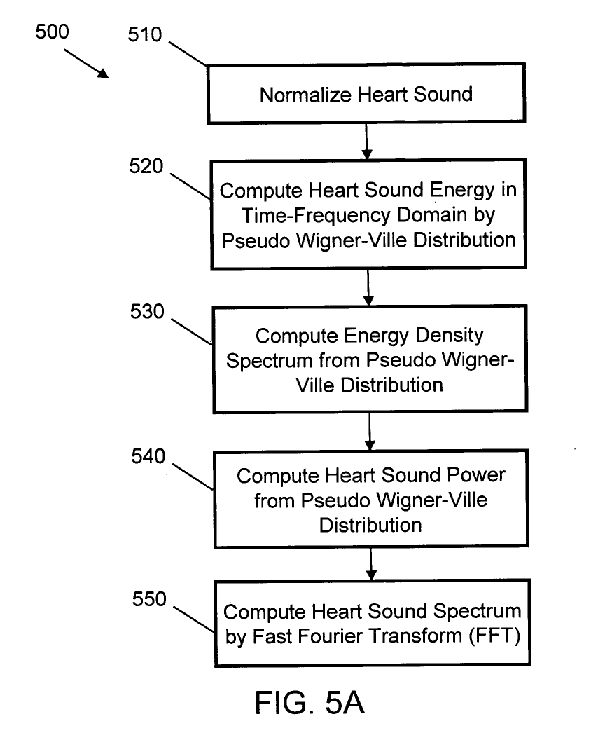

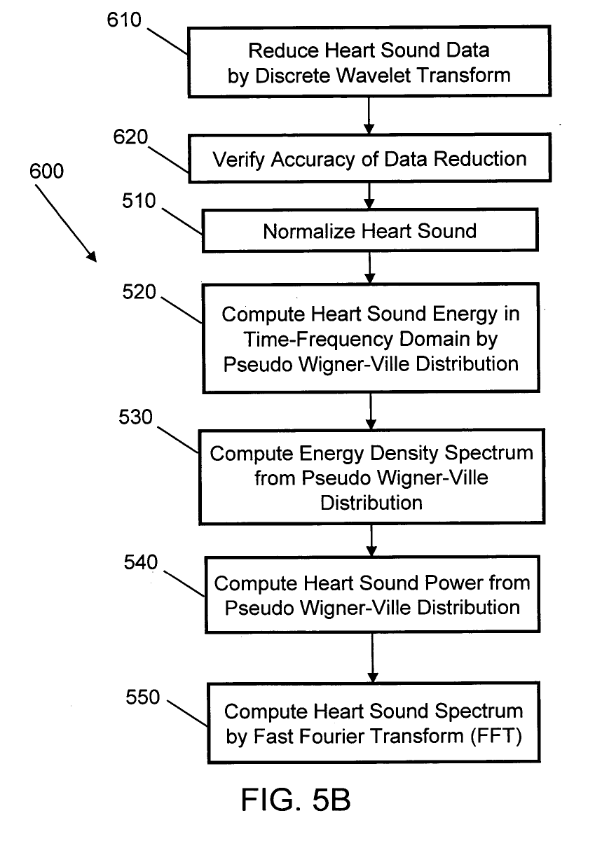

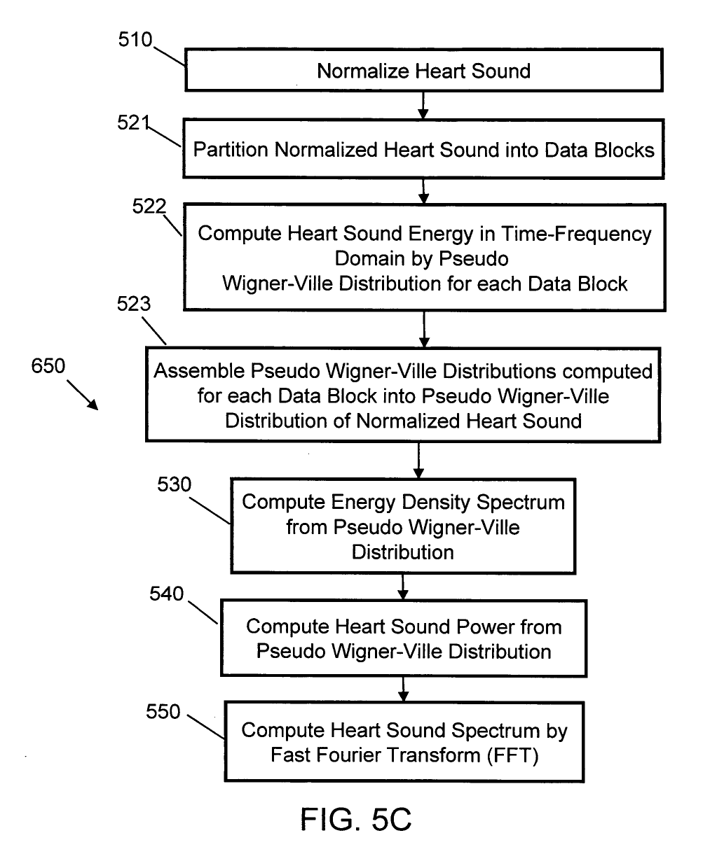

Referring now to FIG. 5A, FIG. 5B and FIG. 5C we present three possible embodiments of the algorithmic scheme for the computation of the heart sound energy signature format 300 that is also defined mathematically by the Equations (1)-(6).

The embodiment referred on FIG. 5A does not require any sample size reduction and can be implemented on an advanced computational systems that do not experience limitations of memory and computational speed. In step 510 we normalize the heart sound signal. By doing so, the cardiovascular sounds obtained from different instruments and measurements could be compared. That is, normalization makes the data instrument and measurement independent. The amplitude of the cardiovascular sounds can vary widely depending on the location of the sensor used and the measurement system (phonocardiograph, electronic stethoscope, etc). Thus, it is very likely that the amplitude of the heart sound for the same patient recorded at the same point will be different depending on the measurement system used.

Frequently, the measurement system can add so-called dc-component (a constant mean value) to the signal, which has no physical significance. This dc-component is removed from the heart sound signal. It is done in two steps. At first the mean value of the heartbeat signal is computed, and secondly this mean value is subtracted from the heartbeat signal. To standardize the comparison of the heartbeat sounds in the time domain, they are normalized to have their amplitude vary between [−1,+1]. The process of normalization of the signal x(t) to [−1,+1] amplitude range is well known in mathematical arts. The basic steps include:
1. find the minimum xₘᵢₙ and the maximum xₘₐₓ values of the signal
2. divide the signal by 0.5*|xₘₐₓ−xₘᵢₙ|
   Presentation of the time signal in the normalized form is important, since the same signal can look differently at different amplitude scales. Furthermore, the normalization of the heart signal by the step 510 creates the signal presentation with easily computed proportionality relationships between the amplitudes of the signal at various time instances.

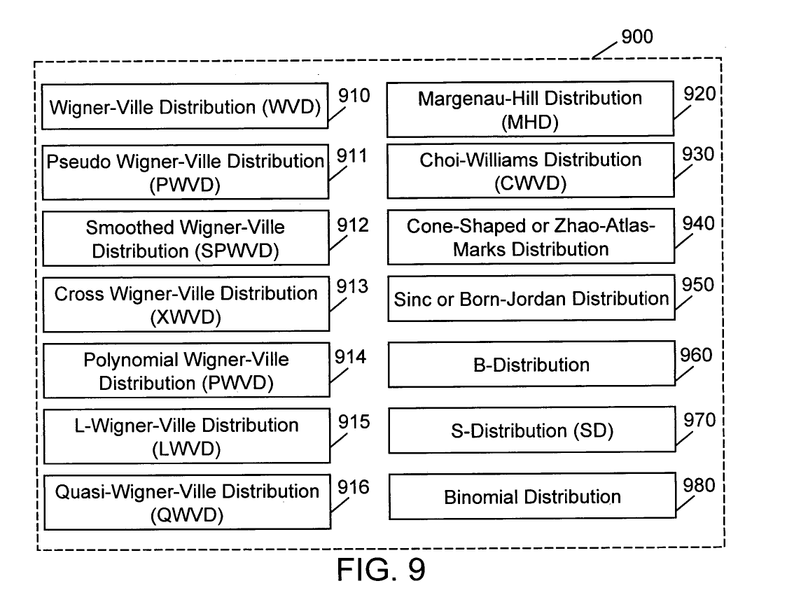

In step 520 we compute heart sound energy in time frequency domain using pseudo Wigner-Ville Distribution (“PWVD”) or any Cohen class type transformation as described in FIG. 9. A special version of the distribution related to the pseudo Wigner-Ville Distribution called S-transformation is described by Stankovic L. and Djurovic I., (“A Note on “An Overview of Aliasing Errors in Discrete-Time Formulations of Time-Frequency Representations”, IEEE Trans. on Signal Proc., Vol. 49, No. 1, pp. 257-259, (2001)); Stankovic L., (“S class of distributions”, IEEE Proceedings: Vision, Image and Signal Processing, Vol. 144, No. 2, pp. 57-64, (1997)); and a higher order L-class transformation is also described by Stankovic L. (” L-class of time frequency distributions”, IEEE Signal Processing Letters, Vol. 3, No. 1, pp. 22-25, (1996)) and is also included in the preferred embodiment and is incorporated herein by reference in its entirety.

An example of utilization of the PWVD type transformation is described in Inventor's U.S. Provisional Patent Application No. 60/546,742, entitled “Heart Energy Signature Description, Method and Format”, filed Feb. 23, 2004, which is incorporated herein by reference in its entirety.

In step 530 we compute energy density spectrum from the above given time-frequency representation. After that step the heart sound power is computed 540 from the time-frequency representation 520. Subsequently the signal spectrum is computed by the FFT 550.

The alternative embodiment as referred in FIG. 5B is designed for the implementation on the computing systems that have specific limitations on the memory and the computing speed. Such systems typically include modern personal computers and laptops, digital organizers or embedded processors. Memory limitations specifically define strong restrictions on the sample size for the transformation, thus limiting heart energy matrix to a very short time interval. Such interval is typically of no use for the diagnosis. In the developed method 600 referred on FIG. 5B we overcome this significant barrier by implementing heart sound data reduction with discrete wavelets 610. Typical down sampling techniques that result in the loss of signal information, such as dropping every 2nd, 3rd, 4th data point are not acceptable here as they lead to a loss in the signal accuracy representation and possible frequency (pitch) alteration. Step of 610 provides significant reduction of the matrix size (up to 3 times from the base frequency of 11 kHz) without much loss in the accuracy. Step 620 will perform the accuracy verification after the data reduction step 610. Subsequent steps follow exactly the sequence defined by 500. Combination of the steps 610 and 620 with the steps 510-550 represents a major breakthrough for the practical reduction to practice and enables an operator skilled in arts to obtain heart energy signatures that include the entire respiration cycle.

Process 650 in FIG. 5C refers to another alternative embodiment of process 500 of FIG. 5A. The process 650 shows block-by-block computation of the heart sound energy signature format 300. A data block with less number of samples than a normalized heart sound is used in this process. The normalized heart sound obtained in step 510 is partitioned into consecutive data blocks in step 521. The consecutive data blocks can be chosen to overlap each other. Then, heart sound energy is computed in step 522 for each data block. The results of these computations are assembled in step 523 into a heart sound energy of the complete normalized heart sound. The following steps 530, 540, and 550 are identical to those used in the processes 500 and 600.

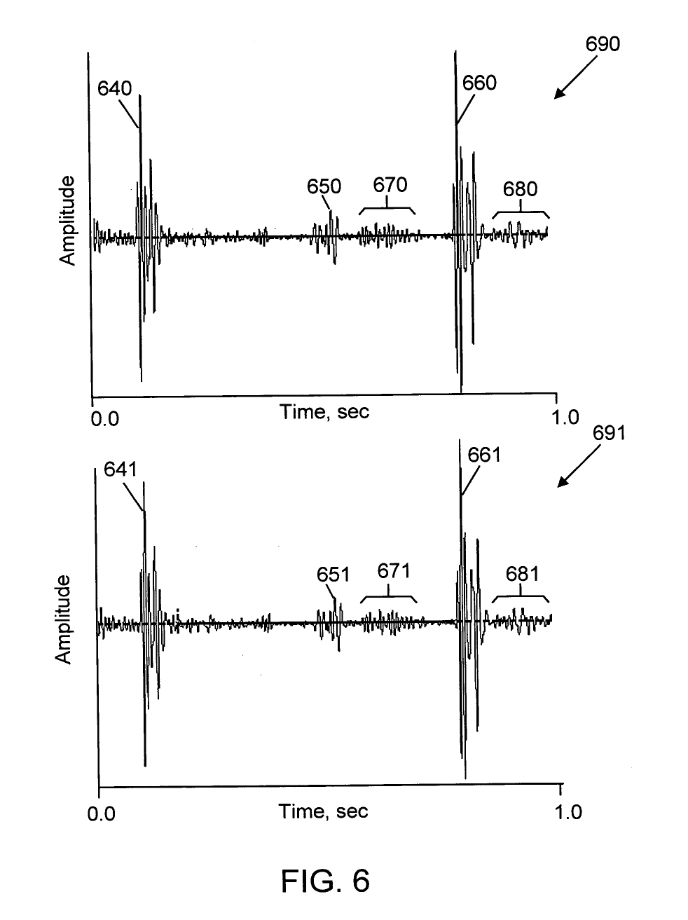

As referred in FIG. 6 original heart sound signal 690 and reduced heart sound signal 691 (four times reduction in number of data samples after two consecutive downsamplings) are compared and the use of the data reduction process 610 is illustrated. Plot 690 presents the segment of actual heart sound signal recorded at 11 kHz sampling rate. Characteristic elements are identified as S1 650 and 651, S2 640 and 641, second split S2 660 ad 661, diastolic murmur 680 and 681, systolic ejection murmur 670 and 671. Step 620 as referred in FIG. 5B verifies the accuracy of the data reduction. The person skilled in art can immediately verify the accuracy of the data reduction by observing the very close similarity between the plots shown in 690 and 691. Accuracy of the data reduction can also be verified by implementing a variety of methods known in the mathematical science to establish a criterion for signal matching and to characterize possible loss of data.

An example of utilization of the data reduction method is described in Inventor's provisional patent application, U.S. Pat. No. 60/546,742, entitled “Heart Energy Signature Description, Method and Format” and filed Feb. 23, 2004, which is incorporated herein by reference in its entirety.

To provide an accurate diagnosis of the heart conditions we must provide an adequate resolution of the signal properties in time and frequency. In this exemplary embodiment the heart sound energy is computed using joint time-frequency distribution belonging to the Cohen class 520 of distributions. The joint time-frequency distribution reflects the distribution of the signal energy in the time-frequency plane. However, the joint time-frequency distribution may not mathematically satisfy the energy properties, i.e. to be positive throughout the time-frequency plane. This is the case with the majority of the time-frequency distributions used in research and referenced in the “Related Art” section. For this invention a joint time-frequency transformation is selected and modified to satisfy the energy properties. In order for the distribution to have the same properties as the energy, the chosen distribution has been modified to be a real positive value at each point of the time-frequency plane.

The steps in obtaining such distribution 520 are outlined below.

A large number of time-frequency distributions of a signal x(t) is given by Cohen's class as

$$C ( t , f ) = \frac{1}{2 π} \int \int \int ϕ ( θ , τ ) x ( t + \frac{τ}{2} ) {x}^{*} ( t - \frac{τ}{2} ) {ⅇ}^{- j θ t - j 2 π f τ - j θ u} ⅆ τ ⅆ u ⅆ θ , \qquad ( 7 )$$

where t is the time, f is the frequency and τ is the running time. The function φ(θ,τ) is the kernel defining the distribution properties. If the kernel φ(θ,τ)=1, we obtain the Wigner-Ville Distribution (WVD):

$${WVD}_{xx} ( t , f ) = \frac{1}{2 π} {\int }_{- ∞}^{∞} x ( t + \frac{τ}{2} ) {x}^{*} ( t - \frac{τ}{2} ) {ⅇ}^{- j 2 π f τ} ⅆ τ , \qquad ( 8 )$$

According to Cohen, L. (“Time-Frequency Distributions—A Review”, Proceedings of the IEEE, vol. 77, No. 7, pp. 941-981, (1989)), the WVD can be regarded as theoretically optimal in that it satisfies a maximum number of desirable mathematical properties. It is shown in the field of the signal processing that all time-frequency distributions of Cohen's class can be computed using a convolution of the Wigner distribution with a two-dimensional impulse response function, as described for example by Mertins A. (“Signal Analysis. Wavelets, Filter Banks, Time-Frequency Transforms and Applications”, John Wiley & Sons, (1999)).

For the kernel φ(θ,τ)=,μ(τ), we obtain the pseudo WVD (PWVD). The Gaussian sliding window function μ(τ) is used because it has an optimal time-frequency concentration:

$${PWVD}_{xx} ( t , f ) = \frac{1}{2 π} {\int }_{- ∞}^{∞} x ( t + \frac{τ}{2} ) {x}^{*} ( t - \frac{τ}{2} ) μ ( τ ) {ⅇ}^{- j 2 π f τ} ⅆ τ , \qquad ( 9 )$$

$$μ ( τ ) = h ( \frac{τ}{2} ) {h}^{*} ( - \frac{τ}{2} ) , \qquad ( 10 )$$

h(τ)=A exp(−σ²τ²), (11)
where A and σ are real positive constants.

The WVD and PWVD are not necessary a positive functions at each point on the time-frequency domain for general signals. From the energy concept, it would be more convenient to work with a positive function as in the case of the magnitude of Fast Fourier Transform (FFT). The WVD can be artificially made positive by simply calculating its absolute value at each point. It also allows the common interpretation of the WVD as an energy density or intensity of a signal simultaneously in time and frequency.

Since for general signals, the WVD takes on negative values, the absolute positive form |PWVDₓₓ(t,f)| of the PWVD is used in the format for the signature of the heart sound. This guarantees the distribution to be positive in the time-frequency plane and makes the straightforward interpretation of the distribution as the signal energy in the time-frequency.

The absolute positive form of the PWVD is used for computation of the heart sound energy distribution 310 defined in step 520 as follows:
E(t,f)=|PWVDₓₓ(t,f,A,σ)|, (12)
where A=1.0, σ²=10⁻⁵, tε[τ,τ+T], fε[f₁,f₂].

The description of the PWVD implementation is described in the Appendix A of the Inventor's U.S. Provisional Patent Application No. 60/546,742, entitled “Heart Energy Signature Description, Method and Format”, filed Feb. 23, 2004, which is incorporated herein by reference in its entirety.

The Wigner-Ville distribution satisfies the frequency marginal condition as specified by Claasen, T. A. C. M., and Mecklenbrauker, W. F. G. (“The Wigner Distribution—A Tool For Time-Frequency Signal Analysis, Part I: Continuous Time Signals”, Philips Journal of Research, Vol. 35, No. 3, pp. 217-250, (1980)).

$${\vert X ( ω ) \vert}^{2} = \frac{1}{2 π} {\int }_{- ∞}^{+ ∞} {WVD}_{xx} ( t , ω ) ⅆ t , \qquad ( 13 )$$

where |X(ω)|² is the energy density spectrum, and ω=2πf is the circular frequency. This equation means that the integral of the WVD over the time variable at a certain frequency ω yields the energy density spectrum of x(t) at this frequency. This property of the WVD is expanded here to compute the energy density spectrum of the heart sound energy signature format (element 370 of the format).

$$D = {\vert X ( f ) \vert}^{2} = {\int }_{τ}^{τ + T} \vert {PWVD}_{xx} ( t , f ) \vert ⅆ t \qquad ( 14 )$$

The WVD also satisfies the time marginal condition

$${\vert x ( t ) \vert}^{2} = \frac{1}{2 π} {\int }_{- ∞}^{+ ∞} {WVD}_{xx} ( t , ω ) ⅆ ω . \qquad ( 15 )$$

This means that the integral of the WVD over the frequency variable at a certain time t yields the instantaneous signal power at that time. Using the energy density interpretation of the PWVD, the signal energy at time t and frequency f contained in a cell dt by df can be found as |PWVDₓₓ(t,f)|dtdf [10]. Other important signal characteristics that can be defined from the PWVD include the instantaneous energy of the signal, or signal power

$$P ( t ) = \int \vert {PWVD}_{xx} ( t , f ) \vert ⅆ f . \qquad ( 16 )$$

Thus, the instantaneous energy of the heart sound signal, or the heart sound signal power 350, is computed as

$$\begin{matrix}P ( t ) = {\int }_{{f}_{1}}^{{f}_{2}} \vert {PWVD}_{xx} ( t , f ) \vert ⅆ f , & t ∈ [ τ , τ + T ]\end{matrix} \qquad ( 17 )$$

The heart sound energy distribution 310 defined and computed by Equations (9)-(11) has a unique correspondence to the input signal, i.e. a pseudo-periodic portion of the heart sound signal. This is a crucial fact in order to obtain the characteristic signature of the signal. One of the popular time-frequency representations previously used in the heart sound research, called short-time Fourier Transform (STFT), does not qualify as a computational method for the signature of the heart sound energy. The equation for STFT is given by

$${STFT}_{x} ( t , f ) = {\int }_{- ∞}^{+ ∞} x ( t ) h ( t - τ ) {ⅇ}^{- j 2 π ft} ⅆ t \qquad ( 18 )$$

where h(t) is the analysis window function. The transform given by the Equation (18) with the Gaussian window function is called Gabor Transform. Since the STFT is complex-valued in general, the spectrogram is used for display purposes. The spectrogram is computed as the squared magnitude of the STFT:

$${S}_{x} ( t , f ) = {\vert {STFT}_{x} ( t , f ) \vert}^{2} = {\vert {\int }_{- ∞}^{+ ∞} x ( t ) h ( t - τ ) {ⅇ}^{- j 2 π ft} ⅆ t \vert}^{2} \qquad ( 19 )$$

Good time resolution of the STFT requires short-duration analysis windows h(t), whereas good frequency resolution necessitates long-duration windows. Frequently the researchers skilled in art simply select by trial and error the window function and its parameters (length, for example) for a particular signal, for example as it was done by Bentley P. M., et al., (1998), “Time-frequency and time-scale techniques for the classification of native and bioprosthetic heart valve sounds”, IEEE Trans. Biomed. Eng., Vol. 45, No. 1. This leads to ambiguity in the time-frequency resolution to the point that two STFT computed for the same signal, but with different window function parameters, could hardly be identified as computed for the same signal. In case of the cardiovascular sounds, as referred in FIG. 6 for example (S1 waveform 650 and S2 waveform 660), when the frequency of the signal quickly changes in time, STFT simply can not well satisfy both time and frequency resolution.

The heart sound energy signature representation referred in FIG. 1 and in FIG. 2C, and its format as referred in FIG. 3 and FIG. 4 provides a method and system for clinical cardiology and heart auscultation practices. It captures simultaneously time, frequency and energy characteristic of a heartbeat and is very simple to understand and to interpret. One skilled in the art can clearly identify closed contour or blob of the energy distribution associated with the sound (elements 30, 50, 40 and 60 of FIG. 1), it bounds in frequency and time, location of maximum energy peak and to compare energies of the various sound components.

Typical format for the signature of the heart sound energy consists of a combination of the images or data sets: two-dimensional image or data of energy, one-dimensional plot or data of normalized heart sound and one-dimensional plot or data of the instantaneous heart sound energy (power). Typical heart sound plot is shown as amplitude versus time signal and is limited just to this information. The signature of the heart sound energy is given by the format 300 and referred in FIG. 3 and it carries out significantly more information about heart conditions of the patient than the original sound itself.

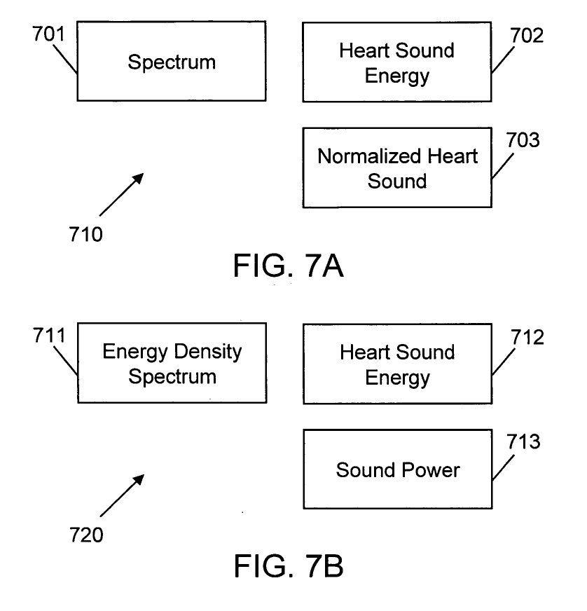

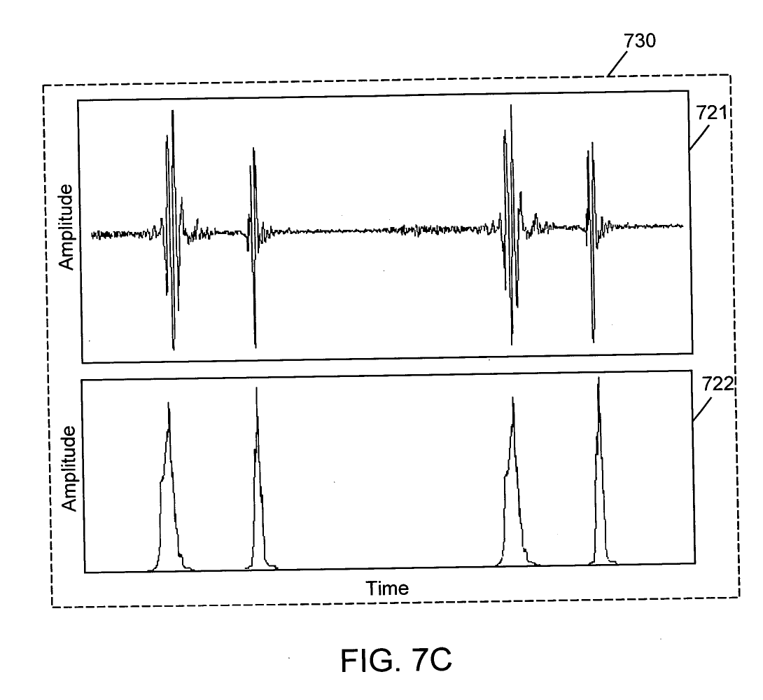

In this exemplary embodiment two display options are outlined. Display option 710 as referred in FIG. 7A includes FFT signal spectrum 701 and heart sound energy signature image 702. Normalized heart sound plot 703 is located right under the energy image 702 to provide maximum visual correspondence between the displays 702 and 703. This correspondence is critical for quick interpretation of the energy signature format 300. As referred in FIG. 7B option 720 presents heart sound energy image 712, heart sound energy density spectrum plot 711 and sound power plot 713. Combination of displays 712 and 713 provides an enhanced ability to quickly identify heart sound segments (heart beats), signal splits, and any additional cardiovascular sounds and murmurs.

Since filing the Inventor's U.S. Provisional Patent Application No. 60/546,742, entitled “Heart Energy Signature Description, Method and Format”, filed Feb. 23, 2004 both of the above referred display options 710 and 720 were successfully reduced to practice in the commercial software product BSignal manufactured by Biosignetics Corporation.

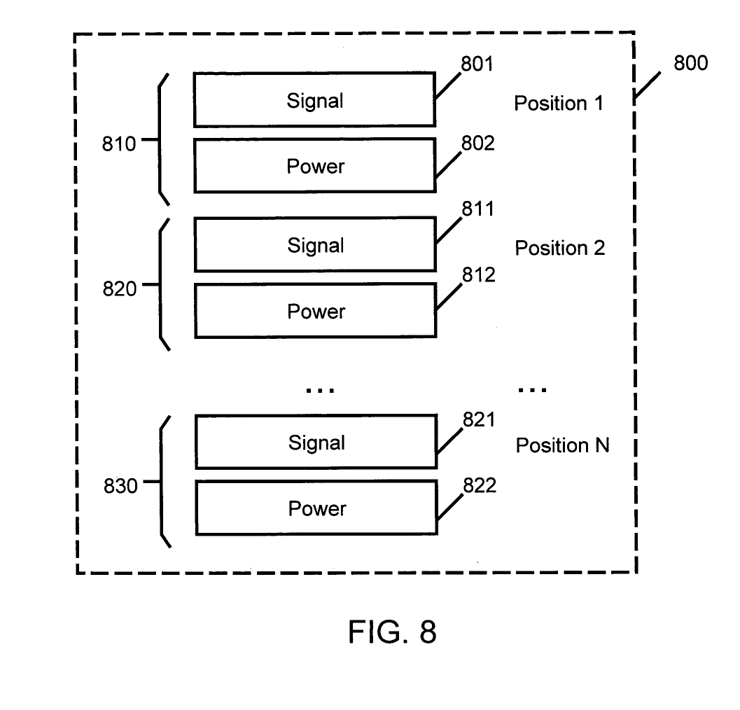

An alternative embodiment 730 as referred in FIG. 7C may include the display of the segment of the heart sound signal 721 and matching display of the signal power 722 obtained from the heart sound energy plot 702. This embodiment will also aid in identification of direct correspondence between the heart sound signal components and matching heart sound energy power distributions. Another example of the alternative embodiment 800 as referred in FIG. 8 aids in simultaneous assessment of the data and corresponding energy signatures obtained for the plurality of N different auscultation positions 810, 820 and 830, where position 830 describes the Nth position. Multiple plots are utilized to simultaneously capture the differences in heart sound signals from several auscultation positions and includes a combination of signal 801, 811 and power 802, 812.

FIG. 9 shows presently known alternative embodiments 900 to the method defined by Equations (9)-(11) and described as step 520 as referred to FIG. 5A. The alternative embodiments 900 are identified as derivatives of the Cohen class time-frequency distributions 910, 911, 912, 913, 914, 915, 916, 970, 920, 930, 940, 950, 960 and are closely related in terms of outcome distributions.

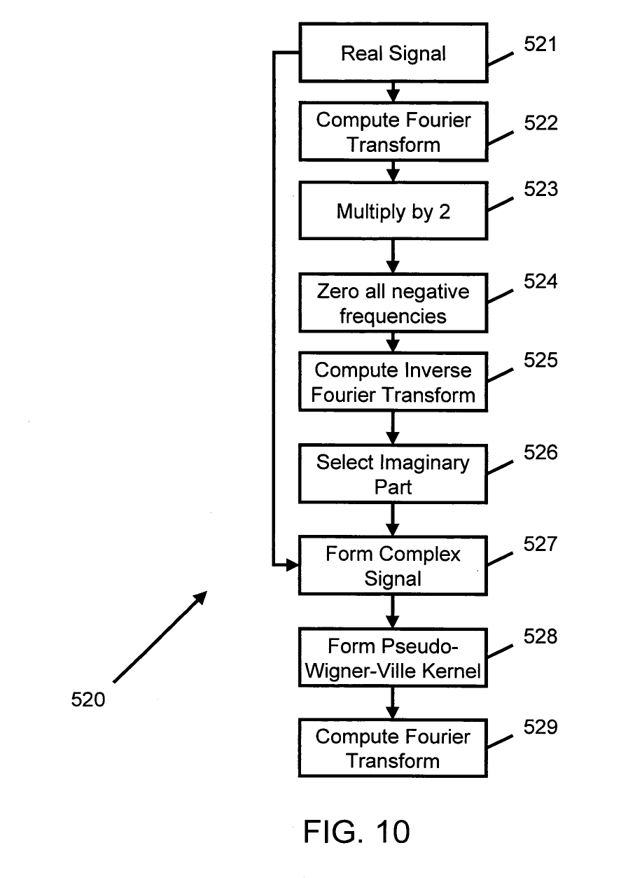

Referring to FIG. 10, the following steps describes in detail step 520 of PWVD computation previously defined by the Equations (9)-(11). Steps 522, 523, 524, 525 describe the process of computation of a Hilbert Transform. Additional steps, 526 and 527 are required to form an analytic signal from the Hilbert Transform. An analytic signal is a complex signal and is obtained in step 527. As a first step in computing a Hilbert Transform, a real signal 521 is obtained and Fourier Transform of the real signal is computed in step 522. It is subsequently multiplied by a factor of two in step 523, all negative frequencies are zeroed 524 and then inverse Fourier transform 525. The result of step 525 is a Hilbert Transform. An imaginary part of Hilbert Transform is subsequently selected in step 526 and a complex signal (analytic signal) is formed in step 527 using also the input of the real signal 521. Subsequently pseudo-Wigner Ville kernel is formed in step 528. In step 529 Fourier transform of pseudo Wigner-Ville kernel formed in step 528 is computed using FFT method. The real part of the Fourier Transform in step 529 contains PWVD for a single time instant. The steps of process 520 are repeated until PWVD for all time instants is computed.

In many clinical situations physician skilled in art may need to monitor specific patient's progress during his or her post-surgery recovery, and due to the drug treatment, diet or physiological changes due to the stress, exercise etc. To access the heart condition of the patient under observation at each time instant, the heart sound energy signature format 300 can be utilized to provide the heart energy distribution in both time and frequency. It contains both time and frequency data only for the short time duration of the particular measurement, and is referred here as local time and frequency.

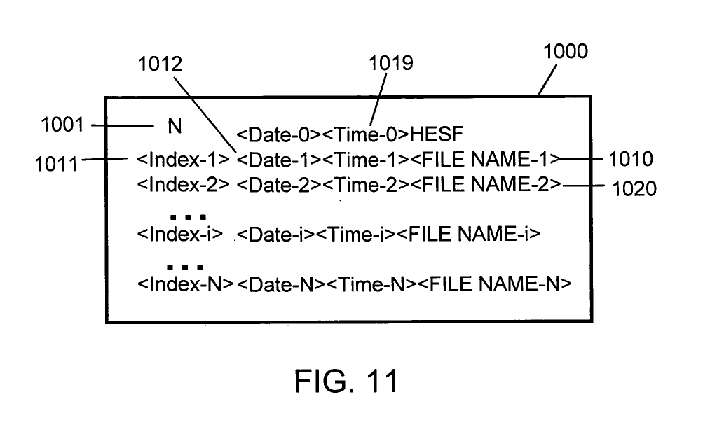

To monitor the historical changes of the signature of the heart sound energy, the format 300 can be expanded to include multiple datasets belonging to the same patient, but made at various times. That is, the patient is monitored, as we refer here, in global time. If the local time refers to a short time span during which the heart sound energy signature format 300 is obtained, the global time refers to any moment in time as is commonly used and understood. The progression of the signature of the heart sound energy can be combined into a single format. This format 1000 is referred on FIG. 11 and consists of many individual elements (slices) that correspond to different global times 1010, 1020, 1030. Total number of slices N is specified as element 1001, index 1011, date 1010, time 1013. The distribution of the heart sound energy simultaneously in local time and frequency, and in global time is given by
E=E(tₐ,t,f), tₐε[t₁,tₙ], tε[τ,τ+T], fε[f₁,f₂] (23)
where E is the heart sound energy distribution, tₐ is the global time, t and f are the local time and frequency, correspondingly.

The set of the normalized cardiovascular sounds corresponding to the heart sound energy at each local time is given by
X(tₐ)={x(t₁),x(t₂), . . . ,x(tₙ)}, tₐε[t₁,tₙ], tε[τ,τ+T], x(t)ε[−1,+1] (24)

The set of the instantaneous energies of the heart sound signal, or heart sound signal power, corresponding to each local time is given by
P(tₐ)={P(t₁), . . . , P(tₙ)}, tₐε[t₁,tₙ], tε[τ,τ+T] (25)

The set of the energy density spectrums of the heart sound, corresponding to each local time is given by
D(tₐ)={|X(f)|₁², . . . ,|X(f)|ₙ²}, tₐε[t₁,tₙ] fε[f₁,f₂] (26)

As referred now on FIG. 11, the file format for heart sound energy flow signature 1000 is expanded to contain rows of independent datasets (1010, 1020, etc) that correspond to the total number N of records 1001.

To monitor the progression of the signal energy changes, the individual slices of the heart sound energy format are stacked in time progression sequence. The resulting three-dimensional energy distribution represents the signature of the sound energy flow, or flow of the signal energy in global time.

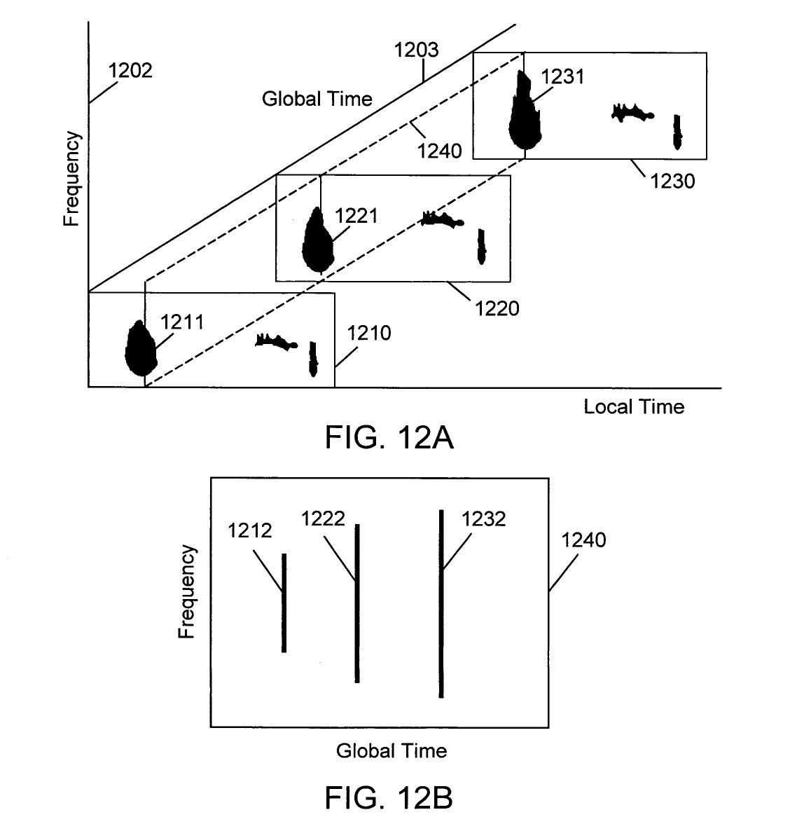

The format of the heart sound energy flow signature 1000 is defined by the Equations (23)-(26) and is constructed using the plurality of the heart sound energy signature formats that are stacked along the X-axis, representing the global time. The axes Y and Z denote the time interval (or, local test time) and the frequency (local frequency), respectively. The rows of data files 1010, 1020, etc. (local instantaneous time-frequency energy plane) in FIG. 11 represent the preferred embodiment for the format of the heart sound energy flow. Referring to FIG. 12A taking the cross-section 1240 along the absolute time axis 1203 creates the plane with the heart energy signature distribution in global time. Obtained in this manner, the time-frequency energy plane 1240 (energy flow plane) allows for an easy visual identification of the signal energy flow (elements 1212, 1213, 1232) in time as referred in FIG. 12B.

As referred in FIG. 12A, heart sound energy flow can be exemplified using three consecutive time slices 1210, 1220, 1230 of the heart sound energy signature plot. On FIG. 12A the frequency axis is identified as 1202 and the global time axis is identified as 1203. Using the representation 1200 we can monitor growth of the S1 energy blob in global time 1203 by picking up the energy instantaneous distributions at local times as shown by elements 1211, 1221, and 1220. The corresponding increase of the sound energy distribution along all frequencies for a given local time instant of the S1 sound with the global time can be monitored on the representation 1240 as referred in FIG. 12B. Changes shown on FIG. 12B indicated changes that happen from test to test over long period of time.

If the signature of the heart sound energy does not change in global time, then the energy flow plane would consist of the signal energy bands of the same width in frequency along the global time. This means that the signal energy frequency bandwidth or signal energy distribution for a given local time is not changing as the time passes. However, even the minor changes in each signature of the heart sound energy at a given local time will be noticed in the energy flow plane 1240 as a deviation of the signal energy from its previous local time-frequency coordinates along the global time. Referring now to FIGS. 12A and 12B, the visual analysis of the heart sound energy flow can be utilized by the person skilled in art for an early detection of the changes in the heart conditions.

An example of utilization of the format of the heart sound energy signature flow and its use for prognostication is described in Inventor's U.S. Provisional Patent Application No. 60/546,742, entitled “Heart Energy Signature Description, Method and Format”, filed Feb. 23, 2004, which is incorporated herein by reference in its entirety.

The visual analysis of the format for the heart sound energy flow can be enhanced by the computation of the differences between the signatures of the heart sound energy and by the computation of the differences between the signal powers obtained at various global times. A set of the heart sound energy signatures can be defined as following
Y(t)={Eᵢ(tⱼ)}, j=1, . . . ,n, t={t₁, . . . ,tⱼ, . . . ,tₙ} (27)
In order to detect the changes that are not obvious by the visual inspection, the following differences can be computed:
dY(tₙⁱ)=Y(tₙ)−Y(tᵢ), where i=, . . . , n−1 (28)

These differences are obtained by subtracting from the most recent signature of the heart sound energy the values of the previously recorded signatures. After subtracting any two signatures, a new energy signature is obtained containing only the residual difference between these heart sound energy signatures. If there are no detectable changes in the heart sound energy signature with global time, then the residual difference signature would contain practically all zero values. This procedure can be generalized as:
dY(tⁱⱼ)=Y(tⱼ)−Y(tᵢ), where for each j=2, . . . ,n, i=, . . . ,j−1 (29)

Similarly, the differences in the heart signal power can be computed and examined for the detection of the changes in the signal power. The equations and the principles are identical to those that were described above by Equations (27)-(29).

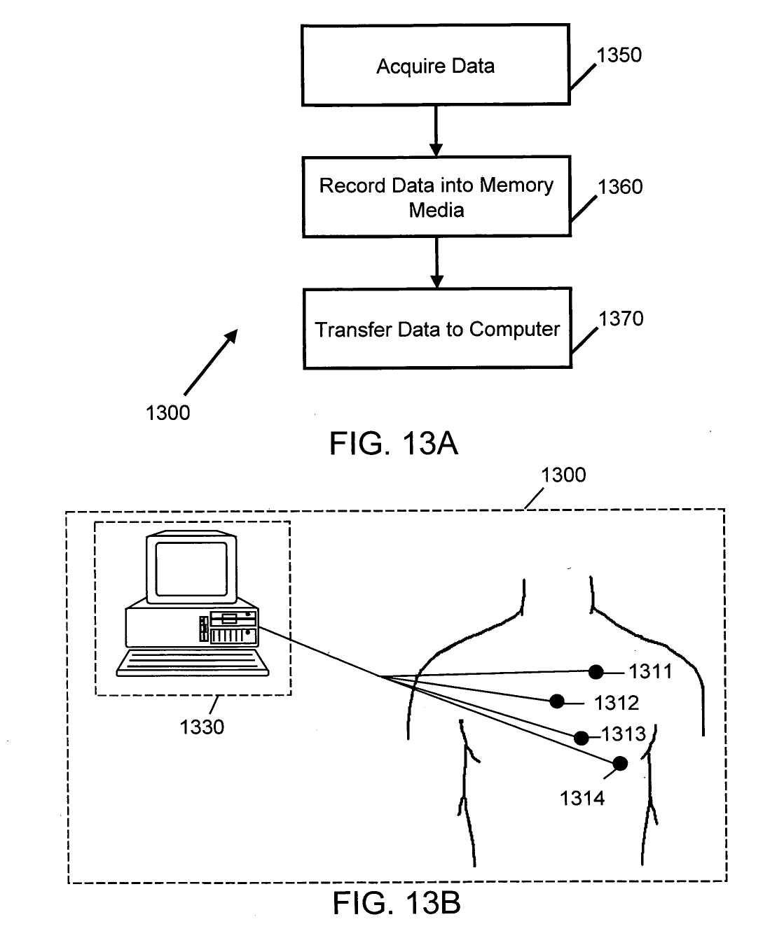

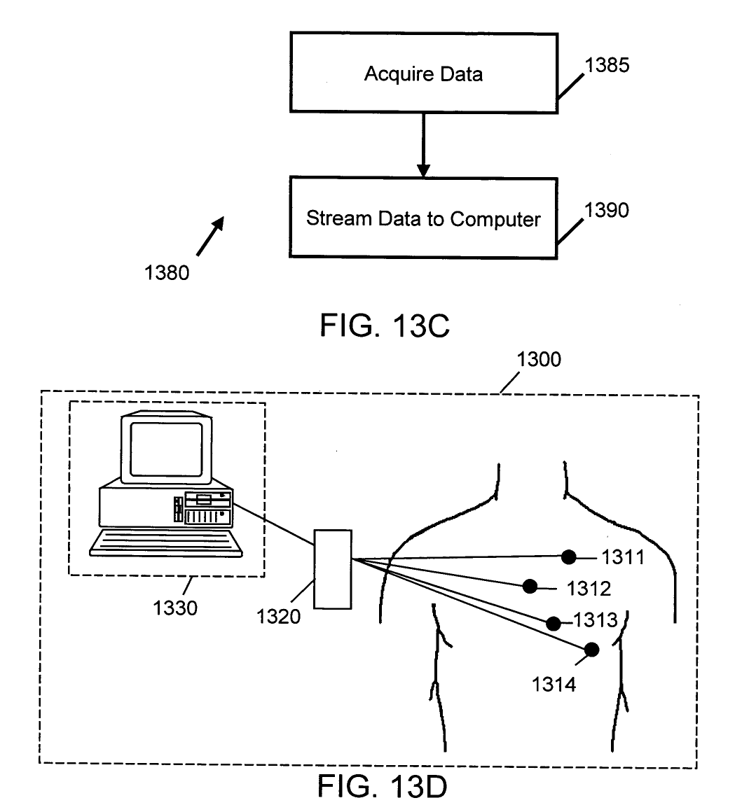

The exemplary embodiments referred in FIGS. 13A and 13B and FIGS. 13C and 13D illustrate two different modes of practical utilization of the heart sound energy signature method, system and format. First, a stand-alone mode of the implementation 1300 is shown in FIGS. 13A and 13B. A stand-alone mode refers to the operation where data are not supplied to a computational or other device in real-time, but already available for the processing on the recordable media. This mode assumes that computer or equivalent device 1303 operates in a stand-alone mode. In this mode the plurality of data sensors 1311, 1312, 1313 and 1314 first deliver sound data to the intermediate memory device 1320 utilizing various methods of data conversion. Subsequently, the data are loaded into the computer for computing and displaying of the heart energy signature and its format. As referred in FIG. 13A, key steps include the data acquisition 1350, recording data into memory in a computer-readable media format 1360 and transferring data to the computing device 1370.

As referred in FIG. 13C, yet another exemplary embodiment include real-time digital or analog circuit where acquired data 1385 are immediately streamed to the computing device 1390. Such implementation will be especially useful for various training and diagnostic systems where real-time information is crucial. It is especially relevant for the current and future implementations of integrated system displays in larger patient monitoring systems or in individual personal monitor gadgets (PDA, etc.) that are capable to operate, transfer and compute data in real time.

Several types of hardware configurations can be utilized in conjunction with the above proposed implementations. The preferred arrangement assumes that data are first collected using electronic stethoscope or pre-amplified acoustic or skin vibratory sensors that are connected to an analog or digital recording device via the converter. Then, data are uploaded from the data collection device to a computer. Then, heart sound energy signature and its format is computed in a stand-alone mode on a computer or processing device. The alternative embodiment may include microphone inserted into the stethoscope tubing with its output connected directly with the sound card of the computer or a digital converter that streams data in real time to a processing device. In this embodiment, the microphone or an alternative sensor is powered and its output is amplified by the sound card or another converter, which also converts the sensors analog output into a digital format. The sound cards are part of the standard configuration for most of the desktop, laptop and palmtop computers.

A combination of the software implementation of the signature of the heart sound energy with the hardware, as described above in 1300 and 1380, creates a new type of cardiometric device—digital stethoscope, which is an alternative to the electronic stethoscopes. It is capable to deliver the good quality cardiovascular sound data to the clinical care, something that existing electronic stethoscopes can not do.

An example of utilization of this exemplary embodiment described in Inventor's paper, entitled “Hemodynamic Pressure Instabilities and their Relation to Heart Auscultation” and published in the Proceedings of ASME PVP Division Conference, 5th International Symposium on Computational Technologies for Fluid/Thermal/Chemical/Stressed Systems with Industrial Applications, Jul. 25-29, 2004, #PVP2004-3126, ASME PVP Vol. 491-2, pp. 113-122, San Diego/La Jolla, USA, which is incorporated herein by reference in its entirety. It is also exemplified in part in the article “Digital Stethoscope” published in the “Movers and Shakers Newsletter” (December 2004), and written by Marcia Freer from the words of the inventors and using the graphic materials provided by the inventors.

Referring again to the FIG. 13B and FIG. 13D, the final point of the data collection process is always a computer or a data processing device 1330, that is capable of implementing methods and systems referred on FIGS. 5A, 5B and 5C.

When cardiovascular sound is collected, it is recommended to follow current standard clinical practices and teachings of the cardiac auscultation. These practices are well described in the following textbooks by M. E. Tavel (“Clinical Phonocardiography and External Pulse Recording”, Year Book Medical Publishers, 2^{nd} Ed, (1976)), S. A. Levine and W. P. Harvey (“Clinical Auscultation of the Heart”, W.B. Saunders Company, (1949)), A. Ravin (“Auscultation of the Heart”, Year Book Medical Publishers, 2^{nd} Ed, (1967)), T. A. Don Michael (“Auscultation of the Heart—A cardiophonetic approach”, McGraw Hill, (1998)) that are incorporated herein by reference in their entirety. Cardiovascular sounds are typically collected in the four zones around the heart—mitral, tricuspid, pulmonary and aortic, but can be collected in any audible area of interest. Sounds are recorded and stored in any analog (audio tape) or digital (wave, mp3, etc) formats in the memory of the recorder or on the recording media (CD, tapes, etc). The analog format is converted into a digital format for processing. Data files are later uploaded or directly copied to the external computer. The signature of the heart sound energy, its format and algorithm is coded as the application software that is executed on the computational device (for example, personal computer, notebook, hand-held computer, microprocessors etc.). This mode of the operation is called stand-alone and is referred on FIG. 13A and FIG. 13B. In this mode, the implementation of the preferred embodiment operates with the pre-recorded heart sound data and no immediate contact with the patient is required.

The signature of the heart sound energy, its format and algorithm can also be coded as software for real or near real-time processing of the heart sound data. In this case, the analog signal representing the heart sound is digitized or converted, then streamed to the computer memory. Then, data is processed directly using at least one of the algorithms outlined on FIG. 5A, FIG. 5B or FIG. 5C. This real-time or near real-time mode of operation is called the direct mode, since it requires a direct data link between the sensor and the software that implements the preferred embodiment. In yet another alternative embodiment the signature of the heart sound energy, its format and algorithm can also be implemented in hardware using electrical circuits, which simulate the operation of the algorithm.

Information processing steps are the foundation for the proper clinical diagnosis. The heart sound energy signature method, system, format and display provide an extended capability to extract very intricate details of the heart operation, to characterize them qualitatively and quantitatively in many different ways. In some cases very limited effort is required and result is immediately obvious, in some cases additional characterization of the energy signature format components referred in FIG. 4A may be required. Operator skillful in art visually identifies repetitive heart pattern by monitoring S1 and S2 signal visual representations in the various displays of the heart energy signature format. This can be any repetitive pattern, but usually S1 and S2 energy areas such as 30 and 40, as referred in FIG. 1A, are the most noticeable due to their high energy.

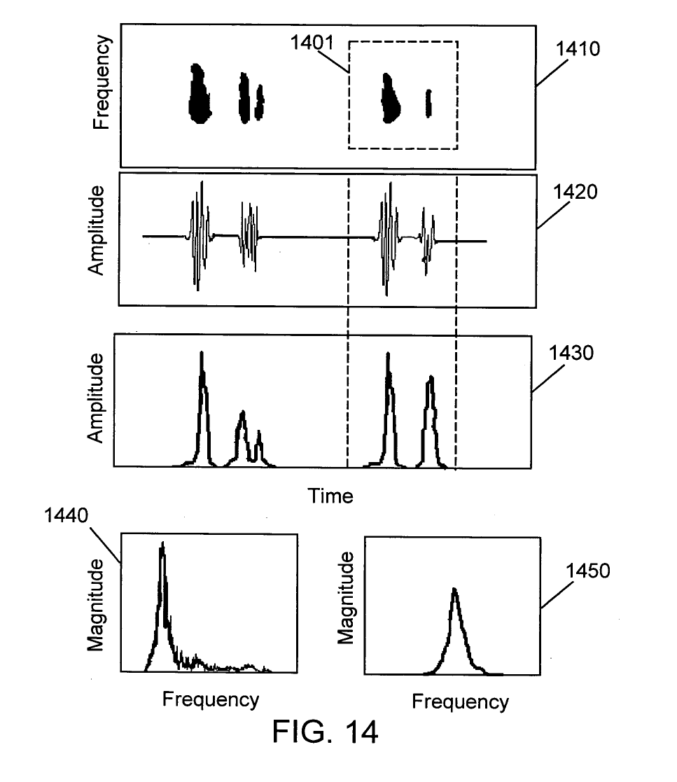

Referring now to FIG. 14, the techniques of the present invention will generally make use of the signature of the heart sound energy image 1410, power plot 1430 and normalized signal plot 1432. Flexible zoom window 1401 allows focusing on a particular detail of the signature of the heart sound energy or power plot or the signal. Additionally power spectral density 1450, and FFT signal spectrum for the pre-selected time window 1440 also aid in forming full characterization of a particular heart sound phenomenon. Signal power 1430 and signal spectral density 1450 can be both presented in time or frequency instantaneous forms. Interactive zoom window 1401 can be applied to study any portion of the heart sound signal, thus, enabling the operator skilled in art to decide on the optimum time interval for the signature of the heart sound energy. This interval is defined to exceed, but to be not less than one heart beat in length.

Referring to FIG. 1, the exemplification of such interval is demonstrated. In the preferred embodiment the operator skilled in art usually utilizes signal plot PCG as referred in FIG. 1 to determine time boundaries 70 and 80 for the heart energy signature. Any number of alternative embodiments can also be implemented by the person skilled in art, for example using heart sound energy image or power plot. Operator can utilize data from any heart energy signature component to decide the time boundaries 70 and 80 of the energy signature and can also decide to document any number of energy signatures of different time durations for the same patient. Such an embodiment allows flexible, but systematic approach to care and to documenting the results, since no patient or heart is the same.

U.S. Pat. Nos. 5,218,969 and 5,025,809 and a textbook (“Auscultation Skills: Breath & Cardiovascular sounds”, (Book with 2 Audio CD-ROMs), published by Lippincott Williams & Wilkins Publishers; 2nd Book and CD-ROM edition, (2002)) and also Audicor method from the Inovise Corporation require that ECG/EKG be performed in parallel with the heart sound collection and be subsequently utilized to recognize heart sound components such as S1 and S2. Present invention does not require ECG/EKG device to simultaneously record electrocardiogram and the phonocardiogram to identify the S1 sound and the beginning and the end of systolic and diastolic periods. This operation can be done by processing the heart sound energy signature and its format.

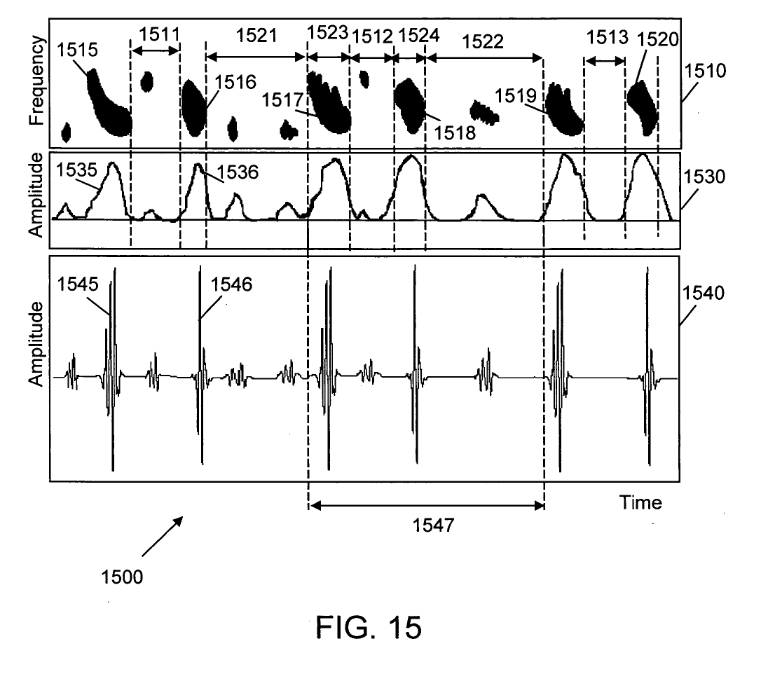

Referring to FIG. 15, a graphical display 1500 of a heart sound signal segmentation principles is illustrated. Heart sound signal segmentation is understood as a process of identification of systolic and diastolic time intervals on the phonocardiogram (PCG) and identification of S1 and S2 heart sound time intervals. This process is absolutely required for the subsequent clinical interpretation of the heart sound and of the heart energy signature and format Sound segmentation utilizes key heart energy signature components such as time frequency sound energy display 1510, integral power display 1520 and heart sound display 1530. Segmentation method allows to establish a unique correspondence between the heart sound display waveforms, their heart sound energy equivalents, to estimate systolic and diastolic periods, and to name appropriate heart sound components (such as S1, S2, S3, S4 and murmurs).

We first establish a direct correspondence between the elements on plots 1510, 1530 and 1540. For example, elements 1515, 1535 and 1545 are located at the same time instant, thus, representing various forms (heart sound energy, heart sound power, heart sound signal) of displaying information about S1 heart sound. Elements 1516, 1536 and 1546 are also at the same time instant, thus, representing various forms of displaying the information about the S2 heart sound.

Subsequently, using the signal power 1520 and signal energy displays 1530 we can establish repetitive patterns of the signals that have highest signal strength. See, for example, 1515 and 1516 (pattern #1), then 1517 and 1518 (pattern #2), and 1519 and 1520 (pattern #3). This process can continue for any pre-defined time duration. Corresponding pattern matches are also established on the signal power plot 1530. Time durations between each element are estimated (1511, 1512, 1513) and time interval lengths of each element are also estimated from 1510 and 1530 (see for example S1 time duration 1523 and S2 time duration 1524. Time intervals that have time duration longer than two adjacent time intervals are marked as diastolic (1521, 1522). Time intervals next to diastolic time intervals are marked as systolic and are marked on FIG. 15 as 1511, 1512, 1513.

Heart sound components that are at the beginning of the systolic interval are identified as S1 1515, and heart sound components that are at the end of the systolic interval are identified as S2 1516. Correspondingly, heart sound components that are to the left of the diastolic interval can be identified as S2 and heart sounds that are to the right of the diastolic interval can be identified as S1.

The differentiation algorithm is based on the fact that S1 and S2 have wider energy spectrum, and considerably higher energy than any other sounds such as murmurs, S1, S4 and clicks. In rare circumstances systolic or diastolic murmurs can be of significant energy and power. In this case triple, quadruple or other arbitrary patterns can be identified.

Person skilled in art can utilize visual or computer based pattern recognition to identify a characteristic single beat pattern component and corresponding elements and time intervals between them. If murmur is systolic then diastolic interval can be utilized to identify S1 and S2, and if murmur is diastolic then systolic interval can be utilized to identify S1 and S2. Heart beat count serves as important check verification for the invented segmentation method. Durations of the S1 1523, S2 1524 and of systolic 1512 and diastolic 1522 periods form one time interval that is equal to the length of a single heart beat 1547. Heart beat duration 1547 can be estimated by a person skilled in art of pattern recognition by the identification of repetitive multi-component patterns on the heart sound energy plot 1510 and on the heart signal power plot 1530.

Alternative embodiments can apply the same logic to conduct heart sound segmentation using the heart sound signal only or heart sound signal in combination with heart energy signature plot (as shown on FIG. 7A) or using heart sound power plot in combination with the heart signal (as shown in FIG. 7C). However, methods that are based on the heart sound signal (1540) can be prone to errors and uncertainties that are due to the noisiness of the heart sound signal and also due to the uncertain visual and signal representation of many different murmurs. This noisiness and uncertainness completely disappear when the heart sound energy 1510 and heart sound power 1530 are utilized for the segmentation method. In rare cases of high intensity murmurs known in cardiology as “Christmas trees” or when S1 sound is very weak, or when the sound is widely split heart sound signal 1540 will be required to confirm segmentation findings, such as for example a comparison of the item 160 with the 175. Item 175 shows two energy blobs identifying split S2, item 160 illustrates signal representation of the split S2 heart sound and confirms that it is in fact split S2 heart sound. Thus, invented segmentation method uses 1510, 1530 and 1540 representations simultaneously. All these representations are derived from the original heart sound.

The following key measurements, characterizations and visualizations are performed in the preferred embodiment:
measuring time intervals between major events (for example 411, 412) on the energy signature display 410, integral 450 and instantaneous power plot 460 and signal plot 420;
determining the pattern of largest duration intervals (1521, 1522) using plots 1510 and 1530 and identifying them as diastolic (exemplified between 1516 and 1517 as referred to FIG. 15);
determining the remaining time intervals 1511, 1512 and 1513 located between the above time intervals as systolic;
determining energy signature peaks (blobs) on the left side of diastolic intervals as S2 (such as 1516 and 1518);
determining energy signature peaks to the right side of diastolic intervals as S1 (exemplified as 1515 and 1517);
identifying additional energy peaks and energy signature components (such as 50 in FIG. 1) in the time intervals between S1 and S2 as systolic events;
identifying additional energy peaks between the S2 and S1 (such as 163 and 164 on FIG. 2C) as diastolic events;
verifying presence of concentrated energy peaks representing diastolic atrial sound, clicks, S3 and S4 sounds by identifying their characteristic frequency, energy, signal intensity, time duration and time position on the heart sound energy signature plot;
verifying presence of concentrated energy peaks representing systolic clicks, snaps or murmurs;
identifying time locations of energy peaks for systolic events and murmurs in terms of their relative position with respect to systolic interval duration;
identifying energy peak locations for diastolic events and murmurs in terms of their relative position with respect to diastolic interval duration;
identifying energy peak values for systolic events and murmurs in terms the relative portion of maximum energy peak value contained within the specific heart beat time interval;
identifying energy peak values for diastolic events and murmurs in terms the relative portion of maximum energy peak value contained within the specific heart beat time interval;
identifying variation in length of diastolic and systolic intervals;
identifying variation in maximum energy on the signature of the heart sound energy 310, maximum signal power 350 and maximum signal amplitude 320;
identifying respiration phases (inspiration-expiration) as a function of heart sound signal strength;
identifying the presence of S2 split (for example elements 172 and 173 in FIG. 2C);
identifying durations of S2 split interval;
identifying S2 split interval dependency on time during the inspiration-expiration cycle and characterizing the nature of this dependency (such as fixed split or time varying split) and values of the time split;
identifying presence of the S1 split and its duration;
characterizing the systolic frequency range and frequency range for each event (from FFT and from the heart sound energy signature);
characterizing diastolic frequency range and frequency range for each event (from FFT and from the heart sound energy signature);
   However, a person skilled in art can also define any number of derivative parameters that can be detected and estimated using the method, format and the system of the invention. The cardiovascular sounds can be collected using various data acquisition methods, at various locations on the patient's body, and in conjunctions with special maneuvers or exercises or even during these maneuvers.

Clinical heart diagnosis using the heart sound is well established art that is now 200 years old. It uses the stethoscope and/or the chart recorded heart sound plots (phonocardiograms) in its foundation. However, they were never used before in conjunction with the heart sound energy signature or its format as defined in the present invention.

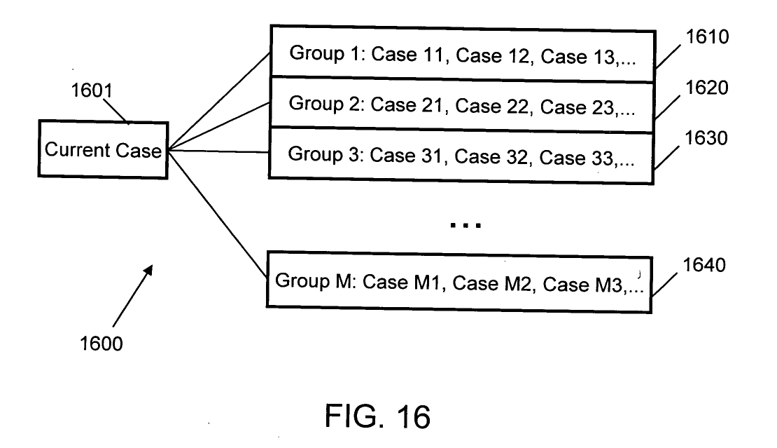

Referring now to FIG. 16, graphical tables of systematically arranged pre-selected heart energy signatures and heart energy signature formats 300 are organized into a computer database system 1600, or into a system of printed matter or folders for the automatic or manual retrieval. It presumes that album or database of pre-selected signatures for normal heart sound and various diseases is available and operator can compare any newly obtained signature with the existing state of the art. It stores all available components of the heart energy signature format in categories typical of various heart conditions known to the person skillful in art of cardiac auscultation.

Images of the heart sound energy signatures, plots and associated materials are stored in groups (such as Group 1 1610, Group 2 1620, Group 3 1630 and Group M 1640) and on case-by case basis within each group (case 11, case 12, etc.) and can be compared against the currently available heart energy signature format components 1601 called “current case”.

Automatic comparison can be enabled through the variety of methods known in the science of image pattern recognition; best match for the signature of the heart sound energy can be recommended automatically or can be sought manually by the operator skilled in the art. Operator starts with checking for number of primary energy blobs such as S1 411 and S2 412 as referred in FIG. 4 and locations, intensities, time durations and frequency ranges of the secondary blobs (murmurs). Database may contain also additional data such as case histories, explanations of disease manifestations, and other useful information. Data can also include heart energy signature formats for several auscultation positions for each corresponding case. Database referred in FIG. 16 can be effectively utilized in the training and education environments, where diagnosis is performed for the training purposes and developing certain clinical diagnosis skills. It can also be useful in the clinical point of care applications when physician may need an additional confirmation or aid in his diagnosis. Such system can be utilized in the stand alone mode in various auscultation training programs. It combines cardiovascular sounds, signal plots and other heart energy signature characteristic elements for the complete characterization of the patient.

### ALTERNATIVE EMBODIMENTS, CONCLUSION, RAMIFICATION, AND SCOPE

While the above description contains much specificity, this should not be construed as limitations on the scope of the invention, but as exemplifications of the presently preferred embodiments thereof. Although the illustrative embodiments have been described herein with reference to the accompanying drawings, it is to be understood that the present system and methods are not limited to those precise embodiments, and that various other changes and modifications may be implemented by one skilled in the art without departing from the scope or spirit of the method and the system. All such changes and modifications are intended to be included within the scope of the invention as defined by the appended claims.

1. A method of processing heart sound signals for storage and display and interpretation of characteristic features, comprising:
obtaining a heart sound signal spanning at least a single heart beat,
generating joint time-frequency distribution of said heart sound signal.

2. The method of claim 1, wherein said heart sound signal is in analog form, further comprising:
sampling said heart sound signal from analog form to a digital form to obtain samples.

3. The method of claim 1, further comprising:
modifying number of samples of said heart sound signal representing at least a single heart beat.

4. The method of claim 1, wherein the step of obtaining said heart sound signal further comprising:
selecting at least a single heart beat from recorded heart sound signal.

5. The method of claim 1, wherein the step of obtaining said heart sound signal further comprising:
recording at least a single heart beat signal.

6. The method of claim 1, wherein the step of obtaining said heart sound signal further comprising:
removing time constant component of said heart sound signal.

7. The method of claim 6, wherein removing time constant component of said heart sound signal further comprising:
computing a mean value of heart sound data,
subtracting from said heart sound data said mean value.

8. The method of claim 7, further comprising:
rescaling heart sound signal amplitude to a predefined range.

9. The method of claim 8, wherein the step of resealing heart sound signal amplitude to a predefined range further comprising:
finding minimum and maximum amplitude values of said heart sound signal,
computing normalization factor equal to a half of the absolute value of a difference between
maximum and minimum amplitude values of said heart sound signal,
reducing said heart sound signal amplitude in proportion to said normalization factor.

10. The method of claim 1, wherein the step of computing joint time-frequency distribution is performed using time-frequency distribution derived to represent energy of said heart sound signal simultaneously in time and frequency.

11. The method of claim 1, wherein the step of computing joint time-frequency distribution is performed using time-frequency distribution derived from a Cohen class of time-frequency distributions.

12. The method of claim 1, further comprising:
generating signal power of a said heart sound signal from said joint time-frequency distribution.

13. The method of claim 12, wherein the step of generating signal power further comprising:
summing up the values of said joint time-frequency distribution corresponding to each time instant.

14. The method of claim 1, further comprising:
computing energy density spectrum of said heart sound signal.

15. The method of claim 12, wherein the step of generating energy density spectrum further comprising:
summing up the values of said joint time-frequency distribution corresponding to each frequency.

16. The method of claim 12, wherein the step of generating energy density spectrum further comprising:
performing a Fourier Transform of said heart sound signal,
computing a magnitude of said Fourier Transform.

17. The method of claim 1, wherein the step of generating joint time-frequency distribution further comprising:
generating joint time-frequency distributions for the parts of said heart sound signal,
assembling said joint time-frequency distributions computed for said parts of said heart sound signal into a joint time-frequency distribution of said heart sound signal.

18. The method of claim 1, further comprising:
making said heart sound signal and said joint time-frequency distribution available for visual inspection by a human operator.

19. The method of claim 12, further comprising:
making said signal power available for visual inspection by a human operator.

20. The method of claim 14, further comprising:
making said energy density spectrum available for visual inspection by a human operator.

21. A method of processing heart sound signals for storage, display, medical diagnostics and prognostics, comprising:
forming a series of joint time-frequency distributions of said heart sound signals,
generating three dimensional array from said series of joint time-frequency distributions.

22. The method of claim 21, wherein the step of forming a series of joint time-frequency distributions further comprising:
obtaining a plurality of heart sound signals,
sorting said plurality of heart sound signals according to their date and time of recording.

23. The method of claim 22, wherein the step of obtaining a plurality of heart sounds is performed so that each of said heart sounds is recorded from the same area of a body.

24. The method of claim 21, further comprising:
displaying said three dimensional array as three dimensional image.

25. The method of claim 21, further comprising:
deriving a joint time-frequency distribution from said three dimensional array of joint time-frequency distributions.

26. The method of claim 25, wherein the deriving a joint time-frequency distribution is performed by extrapolating a sequence of data samples formed from the joint time-frequency distributions of said three dimensional array.

27. The method of claim 25, further comprising:
comparing said joint time-frequency distribution with a reference pattern and feature library, whereby determining if a disease feature is present.

28. A system of documenting heart sound signals, comprising:
means for obtaining a heart sound signal from a living subject,
means for obtaining time-frequency distribution of said heart sound signal,
means for storing said time-frequency distribution of said heart sound signal in computer-readable format.

29. The system of claim 28, further comprising:
means for storing said heart sound signal in computer-readable format.

30. The system of claim 28, further comprising:
means for normalizing said heart sound signal to vary within a predefined range,
means for storing said normalized heart sound signal in computer-readable format.

31. The system of claim 28, further comprising:
means for obtaining an instantaneous signal power of said heart sound signal,
means for storing an instantaneous signal power of said heart sound signal in computer-readable format.

32. The system of claim 28, further comprising:
means for obtaining a density spectrum of said heart sound signal,
means for storing a density spectrum of said heart sound signal in computer-readable format.

33. The system of claim 28, further comprising:
means for obtaining an instantaneous signal power of said heart sound signal for a predefined
frequency of said heart sound signal,
means for storing an instantaneous signal power of said heart sound signal for a predefined frequency of said heart sound signal in computer-readable format.

34. The system of claim 28, further comprising:
means for obtaining a density spectrum of said heart sound signal for a predefined time instant of
said heart sound signal,
means for storing a density spectrum of said heart sound signal for a predefined time instant of said heart sound signal in computer-readable format.

35. A method of determining characteristics events of a cardiac cycle, comprising
obtaining a heart sound signal containing a plurality of heart beats,
generating a joint time-frequency distribution of said heart sound signal,
identifying selected heart sounds and selected cardiac events of a cardiac cycle.

36. The method of claim 35, wherein the selected heart sounds are a first heart sound, S1, and a second heart sound, S2.

37. The method of claim 35, wherein the selected cardiac events of a cardiac cycle are systole and diastole.

38. The method of claim 35, further comprising:
identifying time spans between periodic in time energy blobs within said joint time-frequency distribution of said heart sound signal,
determining the duration of said time spans,
identifying each time span located in-between two shorter in duration time spans as time span corresponding to a diastole.

39. The method of claim 35, further comprising:
generating signal power of said heart sound signal from said joint time-frequency distribution,
identifying signal power zero crossing points corresponding to a beginning and an end of periodic in time energy blobs, within said joint time-frequency distribution of said heart sound signal,
identifying time spans between said signal power zero crossing points,
determining the duration of said time spans,
identifying each time span, located in-between two shorter in duration time spans, as a time span corresponding to a diastole.

40. A database system for managing heart sound signals and diagnostics, comprising:
plurality of data tables, wherein each data table comprises of a plurality of information derived from each of said heart sound signals,
means for creating and adding a data table to said database system,
means of searching and comparing a data table within said plurality of data tables.

41. The method of claim 40, wherein the plurality of information derived from said heart sound signals, comprises:
category identification information for a heart sound signal indicating the health condition of a living subject,
data representing joint time-frequency distribution of said heart sound signal.

42. The method of claim 40, wherein the plurality of information derived from each of said heart sound signals, further comprises:
clinical or diagnostic information for said heart sound signal.
